(19) United States
(12) Patent Application Publication
(10) Pub. No.: US 2024/0139380 A1
(43) Pub. Date: May 2, 2024

(54) FORMULATIONS AND MEDICAL DEVICES FOR MINIMALLY-INVASIVE DEEP TISSUE APPLICATIONS

(71) Applicants: Massachusetts Institute of Technology, Cambridge, MA (US); President And Fellows of Harvard College, Cambridge, MA (US)

(72) Inventors: Keegan Mendez, Cambridge, MA (US); Ellen Roche, Cambridge, MA (US); Connor Verheyen, Boston, MA (US); Jennifer Lewis, Cambridge, MA (US); Markus Horvath, Somerville, MA (US); Sophie Wang, Boston, MA (US); Sebastien Uzel, Cambridge, MA (US)

(21) Appl. No.: 18/312,492

(22) Filed: May 4, 2023

Related U.S. Application Data
(60) Provisional application No. 63/338,285, filed on May 4, 2022.

(51) Int. Cl. A61L 27/52; A61L 27/48

(52) U.S. Cl. CPC A61L 27/52; A61L 27/48; A61L 2400/06; A61L 2430/36

(57) ABSTRACT

Viscoelastic hydrogel microparticles are used for repair of tissue defects and injuries or filling and occlusion of anatomical structures. These are administered as a microparticle suspension using a catheter, syringe, steerable catheter tip, or comparable technology into the site, where they can be further stabilized by crosslinking or sealing, or through incorporation of a support or encapsulating structure. Materials and methods for solidifying, stabilizing and sealing these materials can be used that are also biocompatible and easily deployed with catheters in the body. The micron sized interstitial spacing provides a scaffold for ingrowth and migration of cells into the gel matrices.

### CROSS-REFERENCE TO RELATED APPLICATIONS

This application claims benefit of U.S. Provisional Application No. 63/338,285 filed May 4, 2022, by Massachusetts Institute of Technology and President and Fellows of Harvard College, listing inventors Keegan Mendez, Ellen Roche, Connor Verheyen, Jennifer Lewis, and Sebastien Uzel, which is hereby incorporated herein by reference in its entirety.

### GOVERNMENT SUPPORT CLAUSE

This invention was made with government support under EFMA1935291 awarded by the National Science Foundation and EB015903 awarded by the National Institutes of Health. The government has certain rights in the invention.

### FIELD OF THE INVENTION

This invention is generally in the field of extrusion printing and catheter delivery of viscoelastic hydrogel particles for medical applications.

### BACKGROUND OF THE INVENTION

Biofabrication is an interdisciplinary field that combines engineering, materials science, and biology to build complex three-dimensional (3D) templates for biomedical applications. In the traditional biofabrication workflow, 3D constructs are manufactured and processed in vitro before they are surgically implanted in vivo. Despite decades of scientific progress, few biofabricated therapies have been translated into clinical practice due to outstanding issues related to feasibility, scalability, and logistics. To circumvent these issues, a few groups have recently explored in situ additive manufacturing, where therapeutic scaffolds are sequentially manufactured and processed directly at the site of the damaged or diseased tissue (in situ). While promising, current solutions are limited to superficial applications or small volumes. Further, they display limited capacity for minimally-invasive delivery or patient-specific treatment, which are two key paradigms of contemporary medicine.

Biofabrication uses a combination of tools, techniques, and processes to create engineered templates for tissue repair, reconstruction, or regeneration. The field can trace its roots back to classical tissue engineering, which involves the seeding of cells and bioactive factors on pre-cast 3D scaffolds. As the discipline matured, more advanced techniques emerged, ranging from laser sintering and stereolithography to 3D bioprinting and electrospinning. With the advent of microfluidics and encapsulation, researchers began to pursue modular approaches for highly customized, bottom-up fabrication. Though there is considerable variety in biofabrication technologies, the general approach toward clinical implementation is conserved.

In the conventional biofabrication workflow, 3D constructs are first generated in controlled laboratory environments. After post-fabrication processing and in vitro maturation, finalized constructs are then implanted at a diseased or defective tissue site. Despite decades of consistent scientific progress, very few biofabricated therapies have made it all the way to the clinic. Closing the gap between in vitro biofabrication and in vivo implementation remains a monumental scientific and engineering challenge, with unresolved issues related to feasibility, scalability, and logistics. Further, modern medical practice continues to push for patient-specific treatment and minimally-invasive operations. While this paradigm shift is better for patients, it creates significant technical challenges for biofabrication. Current in vitro biofabrication methods are unable to reliably satisfy the dual design constraints of patient specificity and minimally-invasive delivery.

Methods for in situ biofabrication have been developed. In this approach, an engineered 3D construct is manufactured and processed directly at the site of the damaged tissue, thereby eliminating the lengthy and complicated in vitro portion of the conventional biofabrication workflow. Recently, some groups have tried in situ extrusion bioprinting and in situ digital light processing. Though promising, these techniques are restricted to superficial applications (e.g. dermal), or small homogeneous scaffolds (e.g. microliters) and may not be amenable to patient specificity or minimally-invasive delivery in deep tissue. These restrictions limit the translational potential of these technologies.

A radical new approach is required for personalized biofabrication in deep and poorly accessible tissues with difficult manufacturing environments; requiring a material delivery solution and a multi-dimensional material performance solution to satisfy disparate functional requirements.

It is therefore an object of the present invention to provide more effective methods and materials for biofabrication for treatment of patients, providing methods and materials that are both patient specific and minimally invasive.

It is another object of the present invention to provide a method and materials to use catheter delivery of microgel materials to form volumetric barriers or seals, which may become tissue, in patients in need thereof, thereby avoiding painful and expensive surgery.

It is a further object of the present invention to provide a minimally invasive method and materials that can be delivered through long catheters for treatment and repair of deep, inaccessible tissue defects.

### SUMMARY OF THE INVENTION

Methods and materials have been developed to manufacture and process 3D constructs directly at the site of the damaged tissue, thereby eliminating the lengthy and complicated in vitro portion of the conventional biofabrication workflow, providing a means for personalized biofabrication in deep and poorly accessible tissues with difficult manufacturing environments. (FIG. 1A, 1B) The method uses a combination of a minimally invasive device for delivery of a multi-dimensional formulation, such as microgel particles, most preferably hydrogel particles, to satisfy disparate functional requirements. In a preferred embodiment a microgel-based trans-catheter additive manufacturing method is used for in situ biofabrication. The microgels act as modular building blocks for personalized, bottom-up fabrication, while the long, low-profile catheter system enables minimally-invasive delivery of microgel building blocks to distant tissue locations for in situ additive manufacturing. (FIG. 1C-1F) Advantages of the system is that the catheter can be used to provide means for sealing, such as photo crosslinking and/or a tissue adhesive, as well as for placement of the particles, and patterning of the resulting product. A flexible scaffold can also be incorporated with the microparticles.

Dense, viscoelastic suspensions of hydrogel microparticles are used as bulking agent and for repair of tissue defects and injuries. These are administered as a microparticle suspension using a catheter, syringe, ink printer, or comparable technology into the site, where they can be further stabilized by crosslinking or sealing, or through incorporation of a support structure such as surgical mesh. The technology is particularly useful to reach deep/inaccessible tissues, to rapidly produce stable volumetric materials with good mechanical stability. These materials are particularly advantageous since they achieve a shear-thinning/yield stress profile without using any exotic chemistry or rheology modifiers. Biocompatible microgels act as basic building blocks or modules, and are provided as a biphasic system (liquid to solid), allowing customization/tuning/functionalization of both solid and fluid phases to achieve a desired outcome or material properties, even producing different phases at the same site of administration. Representative ranges include materials having storage moduli 10¹ to 10⁴, narrower range would be 10²-10³, Yield stress 10⁰-10³, narrower range would be 10¹-10², Yield strain 10⁰-10², narrower range would be 10¹-10², Shear thinning exponent ˜0-0.4, narrower range would be ˜0-0.2. Hydrogels provide a high degree of biocompatibility, with many materials already approved for medical use. Hydrogel microparticles have a two to three year shelf life, even at room temperature. The shelf-life will depend on chemical composition and on whether or not it is dehydrated (and reconstituted in the operating room) or stored in liquid. Dehydration yields a longer shelf life but is not preferred in all situations.

Other materials and methods for crosslinking and sealing these materials can be used that are biocompatible and easily used with catheters in the body. The micron sized interstitial spacing of the viscoelastic hydrogel microparticles provides for ready diffusion of nutrients and gases, as well as ingrowth and migration of cells into the gel matrices. These materials can either be replaced by tissue (and degraded) or can remain and become integrated with tissue.

The microparticles can be used for delivery of therapeutic, prophylactic and/or diagnostic agents, including drugs and cells and other biologicals, either encapsulated in the microparticles, suspended with the microparticles, or both.

Hydrogel microparticles, even in large volumes, can be extruded through catheters of various length, diameter, and tortuosity, using methods and devices compatible with existing minimally-invasive routes to target tissues, such as percutaneous and keyhole procedures, and are compatible with mechanical, pneumatic, or manual extrusion approaches. The material undergoes yielding and shear-thinning so it is “liquid-like” during catheter delivery, then at the tissue site it self-heals to recover “solid-like” elasticity using the simple physical jamming/solidifying principle, with no manipulation or processing required to achieve this effect. Studies demonstrate that the methods of applying these materials to a site, including using three-dimensional printing or reversible additive manufacturing, are flexible and omnidirectional, with comparable results even if oriented upside down or sideways, and that the resulting materials have a highly robust mechanical profile, maintaining properties even after months of demanding extrusion testing. Materials may be administered with various volumes and/or into a variety of geometries (planar, curved, convexities/concavities, trabeculations). The materials can be administered using externally controlled or user controlled patterning of material, be removed post administration if needed, and modified in situ. An advantage of administration with a catheter is that the same device can be used to administer materials at the site or multiple sites, then to modify the applied microparticles, for example, by photo-crosslinking using a fiber optic light in the catheter. (FIG. 1C)

The reversible yielding of the dense, viscoelastic hydrogel microparticle suspension provide immediate, solid-like stability. Surface sealing/bulk sealing or encapsulation prevents the microgel scaffold from becoming dislodged when in contact with fluid. There are several ways to achieve sealing, for example, chemical or photocuring. Bulk polymerization can be used to lock the entire scaffold in place. Alternatively, printing into a distensible boundary mesh can be used to prevent microgel migration or escape. A CAD design can be used with the catheter fiber optics to form a specific 3D structure, or a handheld printer can be used to make specific patterns. (FIG. 1I-1J)

The methods and materials, and devices for delivery and processing of the materials have many uses. These include wherein the microparticles are administered to a vein or artery to fill or occlude a site to repair a vascular defect, such as a cerebral, aortic or peripheral aneurysm; wherein the microparticles are used to fill a ventricular or atrial septal defect, such as a left atrial appendage, to form a three-dimensional structure occluding the appendage wherein the microparticles are used to repair a post surgical or obstetrical defect or to form blockages to the passage of urine and fecal matter into the vagina or rectum. Other uses include forming a peri-device occlusion to prevent leakage post implantation of devices such as occluder devices, valves, stents, and flow diverters, for example, wherein the leaks are associated with endovascular coils, endovascular plugs, or transcatheter aortic valve implantation.

Examples demonstrate applications of this technology. In one example microgel microparticles are extruded or printed into explanted tissue defects, where the gel microparticles flow like a liquid through the catheter and into the defect; and solidify into a solid-like three-dimensional viscoelastic microparticle structure when extruded from the catheter, owing to its unique rheological properties; then optionally sealed with a secondary method to form a permanent structure. Another example is the occlusion of a left atrial appendage or septal defect by injection of gel microparticles into the defect to form a three-dimensional structure, which is further stabilized by extruding into an encapsulating casing or mesh to decrease the risk of embolization and increase long term occlusion stability, while still allowing cells to infiltrate to form tissue within the structure. In another example, microparticles are applied to block fistulas, for example, using a cystoscope inserted into the torn areas of the urethra and vagina to form blockages to the passage of urine and fecal matter into the vagina (vesicovaginal fistula) or anus (anal fistula). Other applications include catheter delivery of gel microparticles to form a solidified microparticle three-dimensional structure to repair aneurysms and peri-device leaks, where small and moderate leaks are associated with endovascular coils, endovascular plugs and large leaks is associated with an LAA closure device, or leaks associated with TAVI (percutaneous aortic valve replacement, also known as percutaneous aortic valve implantation, transcatheter aortic valve implantation or transcatheter aortic valve replacement). The technology can also be used with soft robotics approaches to build soft robotic elements in situ.

### BRIEF DESCRIPTION OF THE DRAWINGS

FIG. 1A-1B are schematics of the process of using microgel-based trans-catheter additive minimally-invasive delivery for in situ biofabrication of patient-specific structures using flexible, customizable microgel particles as modular building blocks. FIG. 1A shows the use of a flexible, customizable material for a catheter 10 for minimally invasive delivery of modular building blocks 12 to make patient-specific structures 14. FIG. 1B is a schematic of in situ fabrication 20 of a customizable scaffold 20, 22, 24, using minimally invasive means 26, which is patient 28 specific.

FIG. 1C-1F are schematics of the four types of materials and processing thereof currently available, and the advantages and disadvantages thereof: FIG. 1C shows injection of viscous precursor fluids 30 which are crosslinked in situ 32 to form a scaffold 34; FIG. 1D shows injection 100 of viscoelastic biomaterials 103 which are crosslinked or which “self-heal” to form a scaffold 101; FIG. 1E are schematics showing in situ extrusion bioprinting 106 where viscoelastic biomaterials 108 self-heal/crosslink to form a scaffold 110; and FIG. 1F where viscous precursor fluids 150 are injected 152 then patterned in situ 154 to form a scaffold 166.

FIGS. 1G and 1H and 1I and 1J are schematics of in situ biofabrication (FIGS. 1G and 1H) based on in situ extrusion printing 70 using a handheld printer 72 or injection (FIG. 1I) with catheter or syringe 72 and crosslinking_(FIG. 1J) using patterned light 74 to form a 3D construct 76.

FIG. 2A-2G are schematics of jamming and packaging of microparticles 80 and FIG. 2H-2I show the use thereof in additive manufacturing of microgel-based constructs 82. FIG. 2A are microphotographs of gel microparticle suspension 84 undergoing user-prescribed phase separation, showing un-jammed (free-floating) microparticles 80 (FIG. 2A, 2D) and jammed (densely-compacted) microparticles 86, 88 (FIG. 2B, 2E; 2C, 2F) under increasing pressures to increase volume fraction. FIG. 2D-2F shows the heterogeneity (FIG. 2D), porosity (FIG. 2E), and interlinking (FIG. 2F) of the jammed particles 86. FIG. 2G is a cross-sectional schematic of making solidified microparticles in a syringe 90 to form granular hydrogels 92 for extrusion printing to form a solid structure 92, 94 in a tissue space (FIG. 2H, 2I).

FIG. 2J is a schematic-of biopolymer with photoinitiator and the Type I free radical initiation process that occurs upon exposure to light.

FIG. 2K-2N are schematics of delivery of a curable tissue adhesive (FIG. 2L), polymerization of the surface sealant (FIG. 2M), and stabilized microparticle-based construct (FIG. 2N) for sealing the blood-interfacing surface of the printed construct to mitigate the risk of embolism, shown here in an arbitrary volume and in an excised porcine left atrial appendage. FIG. 2O-2R are schematics of bulk photopolymerization of the “microgel ink” hydrogel particles and photocurable solvent (FIG. 2P), polymerization of the interstitial fluid (FIG. 2Q), and stabilized microparticle-based construct (FIG. 2R), showing stability upon immersion in fluid (FIG. 2O). FIG. 2S-2V are schematics of a mesh enclosure 96 which would encapsulate the microgel ink 98, and prevent embolization of microgel particles, while allowing tissue ingrowth (FIG. 2V) FIG. 3A is a microphotograph of viscoelastic microparticles extruded to form filaments, to fill arbitrary 3D geometries and volumes, in any orientation, which display instant solid-like stability and can be sealed or bulk-polymerized.

FIG. 3B-3M are photographs showing that one can extrude viscoelastic microparticles using a catheter-based delivery system into different configured spaces. FIG. 3C are photographs showing that the viscoelastic microparticles form granular hydrogels which can be filled into complex three-dimensional geometries and volumes from any orientation (bottom to top, FIG. 3B-3M or sideways). FIG. 3N-3Q show that granular hydrogels display instant solid-like stability (FIG. 3N-3P), are stable in water FIG. 3Q) and be sealed (FIG. 3R-3T) or bulk-polymerized (FIG. 3U-3Y).

FIG. 4A-4C are schematics of the process of repairing a three-dimensional tissue defect by extruding from a syringe 101 jammed microparticles 100 in combination with a mesh or casing 102 (FIG. 4A), where the gel 100 is administered with the folded mesh 102 (FIG. 4B), the mesh 102 size prevents particle escape but allows circulation, resulting in stabilization of the defect as cells infiltrate, using the microparticles and mesh as scaffold to form tissue (FIG. 4C) to permanently repair the defect.

FIGS. 5A-5C are schematics of a process of printing gel microparticles into explanted tissue defects, to show that one can place a catheter 104 at the desired site; administer the gel as a liquid suspension 106 (FIG. 5A) into the defect; with sufficient pressure to form a solid-like three-dimensional viscoelastic microspatial structure 110 (FIG. 5B); then seal by photo-crosslinking 108 or surface seal with tissue adhesive to form a permanent structure 112 (FIG. 5C). FIG. 5D shows a schematic of the selection of microparticles that are homogeneous 114, layered mixtures 116, and spatially heterogeneous mixtures 118 for organic specific tissue (connective tissue 120, skeletal muscle 122, epithelial tissue 124, nervous tissue 126, cardiac muscle 128 and smooth muscle 130 formation, as shown in FIG. 5E.

FIGS. 6A-6E are schematics of a modular system 140 for delivery of microgel-based materials compatible with in vivo bioprinting, where the mixture is optimized using a mixture of microgel size, chemistry and shear-thinning properties to produce implants with desired properties (FIG. 6A), which can be delivered using a magnetically controlled steerable catheter 142 (FIG. 6B) to direct the materials then to solidify them, for example, by photopolymerization (FIG. 6C), where the catheter 144 includes a print head 146 that can provide light in a variety of patterns, diameters, and intensity (FIG. 6D) to control crosslinking (FIG. 6E).

FIGS. 7A-7B are prospective schematics of the occlusion of a left atrial appendage 150 by injection (printing) 152 of gel microparticles into the trabeculated tissue appendage 154 (FIG. 7A) to form a three-dimensional structure (FIG. 7B) which is further stabilized by dispersing light through a light diffusing fiber tip to the top of the gel structure to polymerize the gel and thereby decrease the risk of embolization and increase long term occlusion stability 156 (FIG. 7C). FIG. 7D shows sealing 156 of the solidified tissue 158. A glass fiber 166 connected to a light source (405 nm) 168 and a stabilization element using suction, or a balloon can be used for stabilization and/or sealing 174. FIGS. 7E and 7F show how the catheter can be directed into and through the heart 170 (FIG. 7E) for repair of an atrial defect 172 (FIG. 7F).

FIGS. 8A and 8B are cross-sectional schematics of the insertion of gel microparticles using a cystoscope 180 into the torn areas of the urethra or bladder 182 and vagina (FIG. 8A) to form blockages to the passage of urine and fecal matter into the vagina through an anal fistula (FIG. 8B).

FIGS. 9A-9D are cross-sectional schematics of the use of catheter delivery of gel microparticles to form a solid-like microparticle three-dimensional structure to repair peri-device leaks (FIG. 9A, minor PDL; FIG. 9B, small PDL; FIG. 9C, moderate PDL; FIG. 9D, large PDL; where small and moderate are associated with endovascular coils, endovascular plugs and large is associated with an LAA closure device.

FIGS. 10A-10C are schematics of remotely steerable, ferromagnetic catheter printhead and use thereof. Step one is to conduct pre-procedural imaging and 3D reconstruction, then remotely controlled printhead path planning, then in vivo printing under remote actuation with a magnet (FIG. 10A). The ferromagnetic printhead typically includes a catheter tip having ferromagnetic particles embedded in a polymer matrix with programmed magnetic polarity. Steering of this printhead with an external magnet allows controlled printing of the jammed microgel. (FIG. 10B). Light delivered from optical fibers in the catheter provide a means for photopolymerization of the injected particles, sealant or ink solvent.

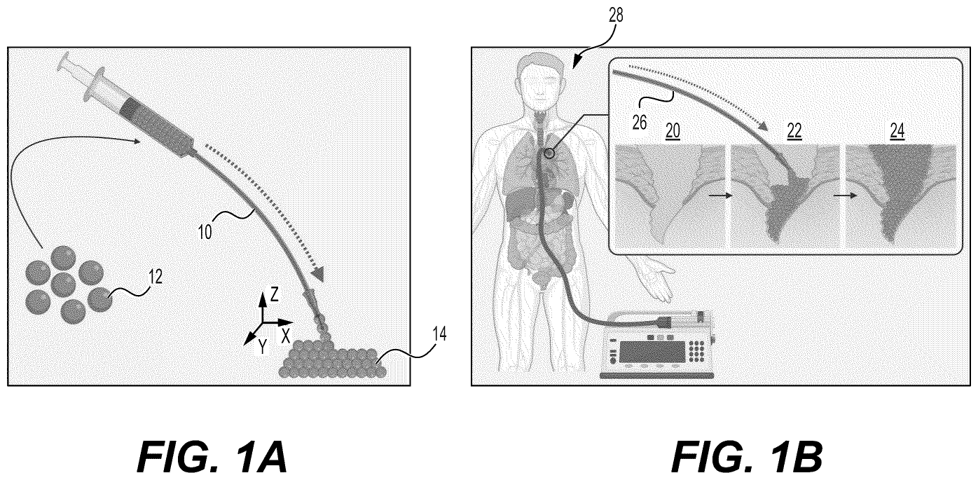

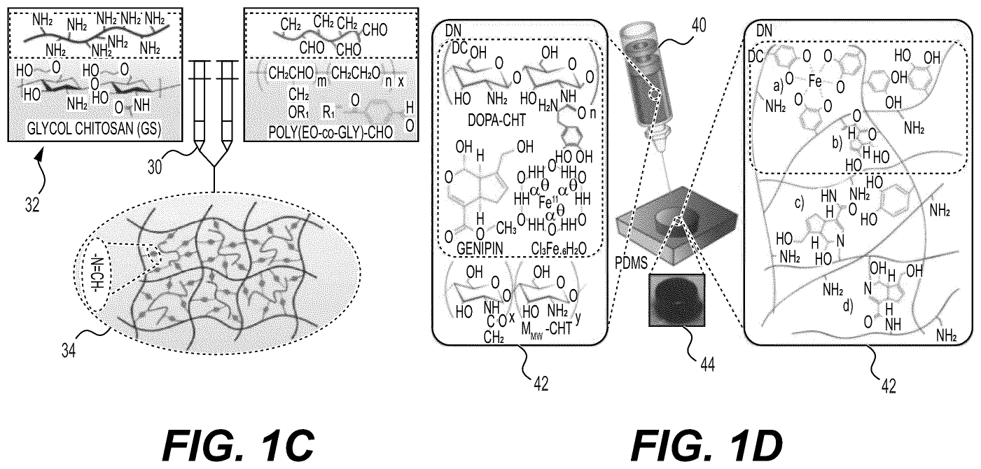

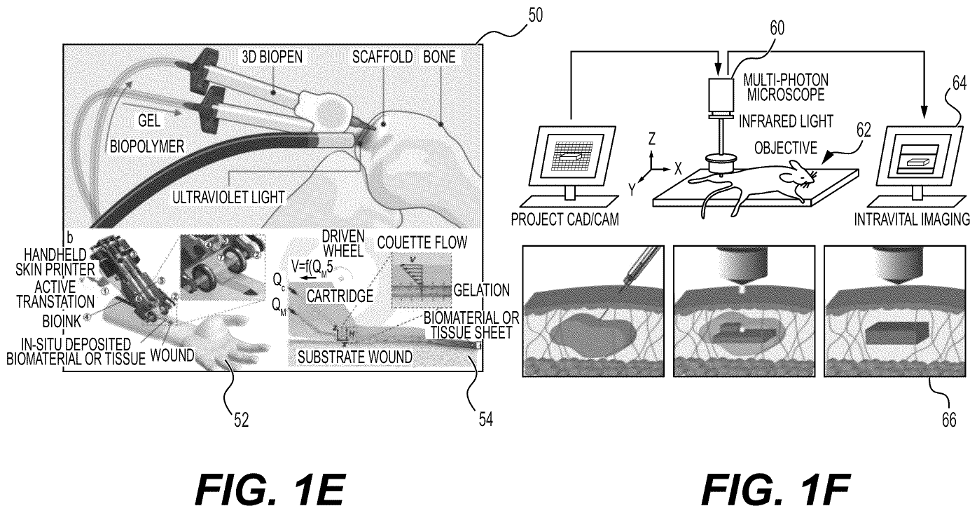

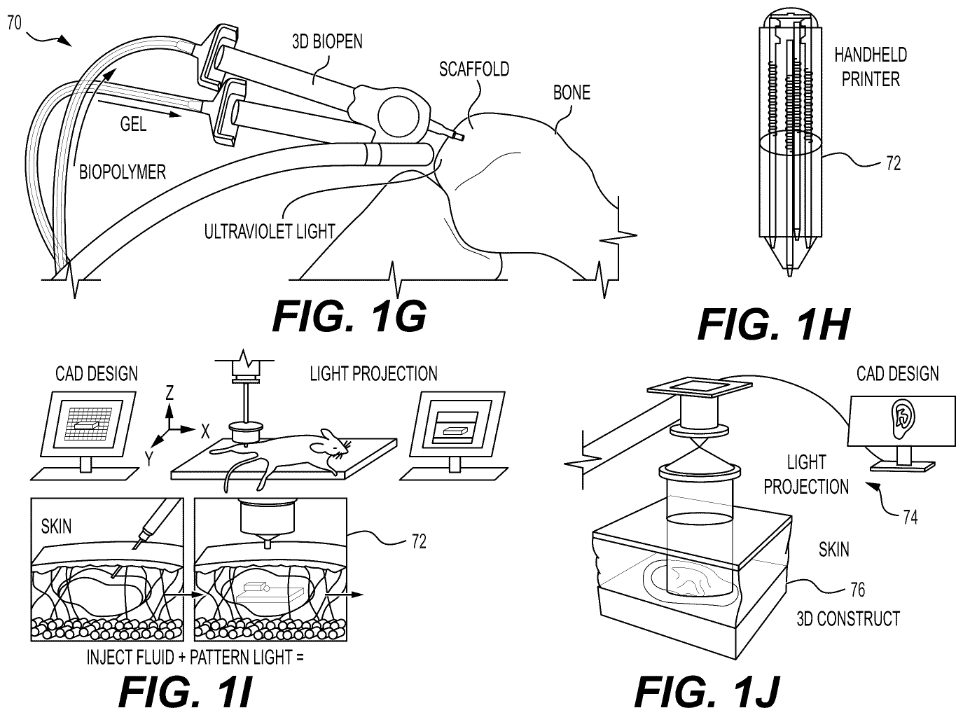

### DETAILED DESCRIPTION OF THE INVENTION

The technologies described herein provide a trans-catheter 3D bioprinting platform enabling customized, biocompatible fabrication at the site of a patient's tissue defect in a minimally-invasive fashion. Bioinks composed of hydrogel microparticles can be printed in situ through a robotic catheter. The mapping of tunable input parameters (e.g., microparticle diameter and fluid phase viscosity) to desired printing outcomes (e.g., shear-thinning profile and rapid elastic recovery) produce design rules for creating bioinks for specific clinical applications. Catheter-based technologies support controlled biomaterial delivery in vivo. Essential design features include navigation to the tissue site, isolation of the defect from surrounding tissue, controlled delivery of the bioink in three dimensions, and stabilization of the bioprinted construct to ensure long-term structural integrity. These technologies provide the means for treating myriad clinical indications with varying tissue properties and anatomic location.

Granular hydrogel microparticles s are a versatile and effective platform for tissue engineered constructs in regenerative medicine. When hydrogel microparticles (HMPs, or microgels) are compacted above a minimum volume fraction, they form a dense granular hydrogel scaffold that displays bulk viscoelastic properties. These injectable, microporous scaffolds possess self-assembling, shear-thinning, and self-healing properties.

The microparticles can be delivered to remotely accessed deep tissue areas in need of treatment using catheters. These may be conventional catheters including optical fibers for photopolymerization; externally manipulatable catheters for placement into areas difficult to reach with conventional catheters, or specialized catheters that include microjet or print heads for deposition and patterning of microparticles in more complex arrangements. 3D printing involves the development of inks that exhibit the requisite properties for both printing and the intended application. In bioprinting, these inks are often hydrogels with controlled rheological properties that can be stabilized after deposition.

The materials and delivery means can be used to treat a wide variety of tissue defects, in different organ systems and tissues, as discussed in more detail below.

### I. Definitions

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable carrier”, as used herein, refers to all components of a pharmaceutical formulation which facilitate the delivery of the composition in vivo. Pharmaceutically acceptable carriers include, but are not limited to, diluents, preservatives, binders, lubricants, disintegrators, swelling agents, fillers, stabilizers, and combinations thereof. In the preferred embodiments herein, the excipient or carrier for the microparticles is sterile water or phosphate buffered saline (“PBS”).

“Effective amount” or “therapeutically effective amount”, as used herein, refers to an amount of drug effective to alleviate, delay onset of, or prevent one or more symptoms of a disease or disorder.

The terms “treating” or “preventing”, as used herein, can include preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

“Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

Hydrophilicity and hydrophobicity can be spoken of in relative terms, such as, but not limited to, a spectrum of hydrophilicity/hydrophobicity within a group of polymers or polymer segments. In some embodiments wherein two or more polymers are being discussed, the term “hydrophobic polymer” can be defined based on the polymer's relative hydrophobicity when compared to another, more hydrophilic polymer.

Hydrogel refers to a crosslinked hydrophilic polymer that does not dissolve in water. They are highly absorbent yet maintain well defined structures.

“Microparticle”, as used herein, generally refers to a particle having a diameter, such as an average diameter, from about 1 micron to about 1000 microns, preferably from about 10 to about 100 microns. The microparticles can have any shape. Microparticles having a spherical shape are generally referred to as “microspheres”.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

As used herein, “viscoelastic” (which includes viscoelastic) refers to a material that will flow under shear but will act as a solid upon removal of the applied shear.

As used herein “deep tissue” refers to a site in the tissue not accessible through the skin or by an injectable, therefore requiring catheter or surgical access.

### II. Viscoelastic Microparticles

Viscoelastic microparticles are used as bulking agent and for repair of tissue defects and injuries. These are preferably hydrogels which are administered as a microparticle suspension using a catheter, syringe, ink printer, or comparable technology into the site, where they can be further stabilized by crosslinking or sealing, or through incorporation of a support structure such as surgical mesh. These materials are advantageous since they achieve a shear-thinning/yield stress profile without using any exotic chemistry or rheology modifiers, nanoparticles etc. The microgels act as basic building blocks or modules. The materials are provided as a biphasic system, allowing customization/tuning/functionalization of both solid and fluid phases to achieve a desired outcome or material properties, even producing different phases at the same site of administration. The hydrogels provide a high degree of biocompatibility, with many materials already approved for medical use.

Materials and methods for crosslinking and sealing these materials can be used that are also biocompatible and easily used even with catheters in the body. The micron sized interstitial spacing provides for ready diffusion of nutrients and gases, as well as ingrowth and migration of cells into the gel matrices.

An example of a biocompatible ink composed of densely-compacted microgels, which are designed to incorporate a range of properties through microgel design (e.g., composition, size) and through the mixing of microgels, is described by Highley, et al. Adv. Sci. 6, 1801076 (2019). The dense and viscoelastic microgel inks are shear-thinning to permit flow and rapidly recover upon deposition, including on surfaces or when deposited in 3D within hydrogel supports, and can be further stabilized with secondary cross-linking.

A. Hydrogel Polymers

Hydrogels are three-dimensional, hydrophilic, polymeric networks capable of absorbing large amounts of water or biological fluids. Due to their high water content, porosity and soft consistency, they closely simulate natural living tissue, more so than any other class of biomaterials. Hydrogels may be chemically stable or they may degrade and eventually disintegrate and dissolve.

Polymers used to form the hydrogels are biocompatible hydrophilic polymers. Examples include natural polymers such as alginate, collagen, chitosan, gelatin, hyaluronic acid, celluloses, and dextran, and synthetic polymers such as block copolymers of polypropylene oxide such as polyethylene glycol (PEG), acrylates and methacrylates, polyvinyl alcohol, and poly(N-isopropylacrylamide). This platform allows the use of microgels engineered from various materials (e.g., thiol-ene cross-linked hyaluronic acid (HA), photo-cross-linked poly(ethylene glycol), thermo-sensitive agarose), and can incorporate cells, where the microgel compaction process and printing do not decrease cell viability.

The polymers are crosslinked to form the hydrogels. These can be ionic and/or covalent crosslinks. Ionic crosslinking is usually by means of addition of divalent ions; covalent crosslinks are often the result of photo-crosslinking.

The material formulation is an inherently biphasic system composed of solid-phase crosslinked microgels with a liquid-phase carrier fluid surrounding the microgels. Like the microgels, this carrier fluid can also be modified or functionalized. For example, carrier fluid manipulation has been used to: 1) fine-tune the gel microparticle attributes and the solid-fluid interactions to alter bulk material properties, 2) ensure adequate shear transmission and recover extrudability for stiff and highly-frictional particle formulations, 3) chemically functionalize the interstitial space and leverage photopolymerization to crosslink the bulk scaffold after delivery, and 4) make the material radiopaque so it can be guided and observed via non-invasive fluoroscopic techniques.

Carrier fluid modifications can include 1) the incorporation of different types of salts, ions, or biomolecules, 2) the incorporation of rheological modifiers and synthetic or natural polymers of varying molecular weight, 3) the incorporation of various chemical groups for additional crosslinking or other secondary and tertiary functionality, 4) the incorporation of contrast agents for medical imaging and non-invasive monitoring. Thus, carrier fluid manipulation can be leveraged to enable: diverse mechanical behaviors, reliable extrudability profiles, follow-up crosslinking and processing steps, and clinically-relevant design features to support translation.

B. Methods of Making Viscoelastic Hydrogel Microparticles and Methods of Crosslinking

Microparticles can be made by methods known to those skilled in the art by crosslinking of polymer in a solution or suspension. Density of polymer solution, nozzle size, distance to solidification, and other parameters are used to control the size of the resulting microparticles. The density is a function of the precursor preparation, synthesis protocol, and post-synthesis processing and solidifying procedure. Particles can be collected by filtration or centrifugation.

As described below, work has been done with ionic gelation of gel building blocks (physical) and photo-initiated gelation of bulk structures (chemical/covalent).

Methods for Hydrogel Crosslinking Include:

Chemical crosslinking methods: Crosslinking by radical polymerization; crosslinking by chemical reaction of complementary groups (aldehydes, addition reactions, condensation reactions); crosslinking by high energy irradiation; crosslinking using enzymes

Physical crosslinking methods: Crosslinking by ionic interactions; crosslinking by crystallization; Physically crosslinked hydrogels from amphiphilic block and graft copolymers; Crosslinking by hydrogen bonds; Crosslinking by protein interactions

Methods are known in the art. See, for example, Hennink, W. E., & van Nostrum, C. F. (2012). Novel crosslinking methods to design hydrogels. Advanced drug delivery reviews, 64, 223-236.

Methods for fabricating hydrogel microparticles include:
Lithography (imprint lithography, photolithography, flow lithography, stop-flow lithography),
Mechanical fragmentation (blender fragmentation, forced mesh fragmentation),
Batch emulsion, Droplet microfluidics (flow-focusing devices, centrifugal microfluidics, in-air microfluidics),
Extrusion methods (simple dripping, electrohydrodynamic spraying, air extrusion)

See, for example, Alzanbaki, Hamzah, Manola Moretti, and Charlotte AE Hauser. “Engineered Microgels—Their Manufacturing and Biomedical Applications.” Micromachines 12.1 (2021): 45.

Commonly used materials are poly (ethylene glycol) diacrylate (PEGDA), gelatin methacrylate (GelMA), collagen methacrylate (CollMA), and hyaluronic methacrylate (HAMA), which are coupled with photoinitiators such as IRGACURE® (365 nm), lithium acyl phosphinate (LAP, 365 nm and 405 nm), ruthenium (visible light) and eosin Y (visible light). Experiments using surface sealants and bulk polymerization yielded microgel-based constructs that were stable indefinitely in fluid-filled environments after completing this secondary stabilization step.

The currently preferred approach for gel microparticle generation uses a simple and scalable air extrusion system. Precursor solutions of alginate are prepared with user-defined attributes (concentration, viscosity, selection of polymers, materials to be encapsulated such as drugs, cells, diagnostic or imaging agent such as fluorescent molecules, etc. Precursor solutions are mechanically extruded through nozzles into airstreams to induce droplet detachment. Post-detachment, the particles are crosslinked in a downstream gelation bath (currently ionic gelation (Ca2+), though other forms of crosslinking can be used). The nozzle types, flow rates, pressures, dimensions, distances, and gelation bath characteristics can all be tuned to modify the particle properties. Post-gelation, the microgels can be collected via settling, filtering, or centrifuging. The dynamic nature of the microgels supports a range of post-processing strategies. For example, the size, stiffness, friction, and opacity of the microgels can be further modulated by exchanging the gelation bath for a variety of other suspending fluids with diverse properties (e.g. differing ion/salt concentrations). For further customization, the fluid phase can also be mechanically modified (e.g. incorporation of rheological modifiers and viscosity enhancer) or chemically modified (e.g. incorporation of photopolymerizable polymers or biodegradable materials).

Microparticles are preferably between greater than 10 and 100 microns, however microparticles up to 1000 microns have been successfully utilized, with the ideal size determined by the application. Granular hydrogels having a diameter greater than 10 μm experience stronger gravitational forces relative to thermal forces. Additionally, the van der Waals force between adjacent hydrogel microparticles is nominal relative to friction. The relatively larger particle size, lack of thermal motion, and existence of friction distinguishes granular hydrogels from other particulate matter, such as colloidal gels. These features also explain why granular materials with a particle-volume fraction above approximately 0.58 (known as “random loose packing”), under sufficient conditions of stress and temperature, display a phenomenon in which the microscopically disordered material has transitioned from “liquid-like” to “solid-like”. When hydrogel microparticles are concentrated above random loose packing, especially closer to a particle-volume fraction of 0.64 (known as “random close packing”), they can collectively be perceived as a bulk entity (i.e., a granular hydrogel scaffold) possessing conventional hydrogel properties. This bulk gel, however, is dynamic in nature where minimal external force can reorganize and displace particles.

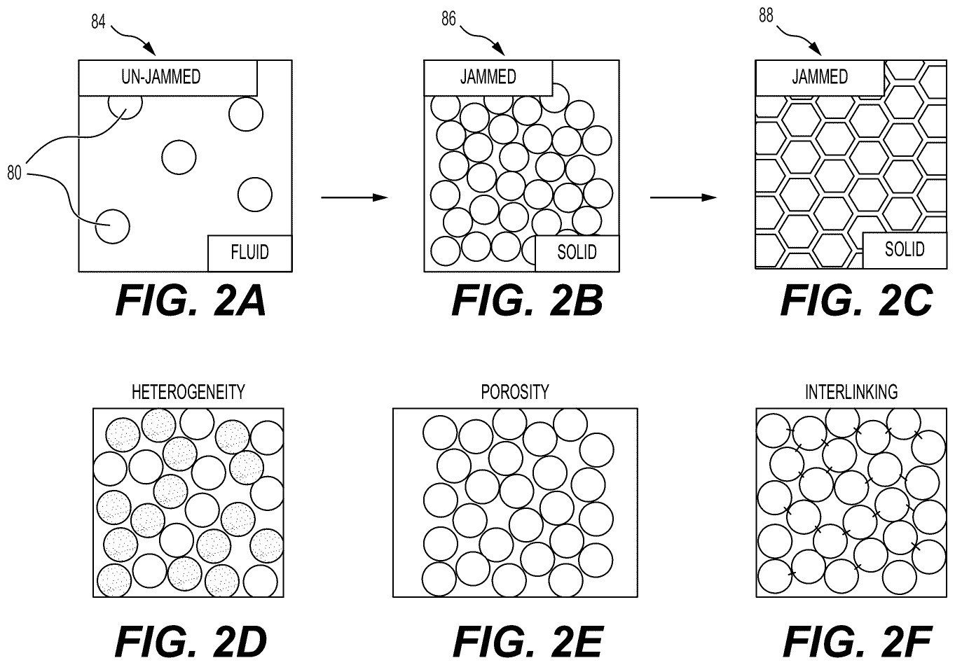

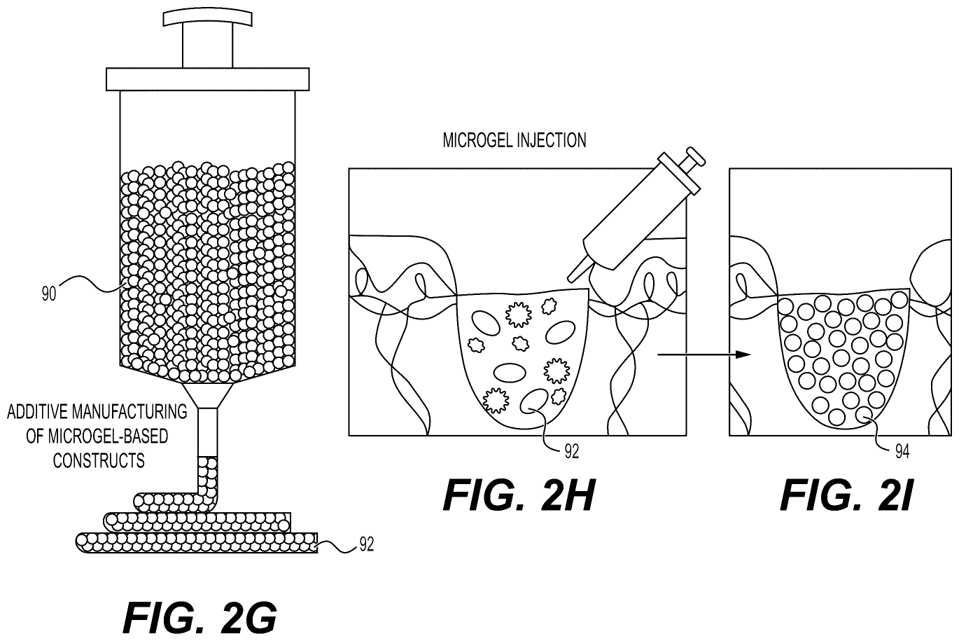

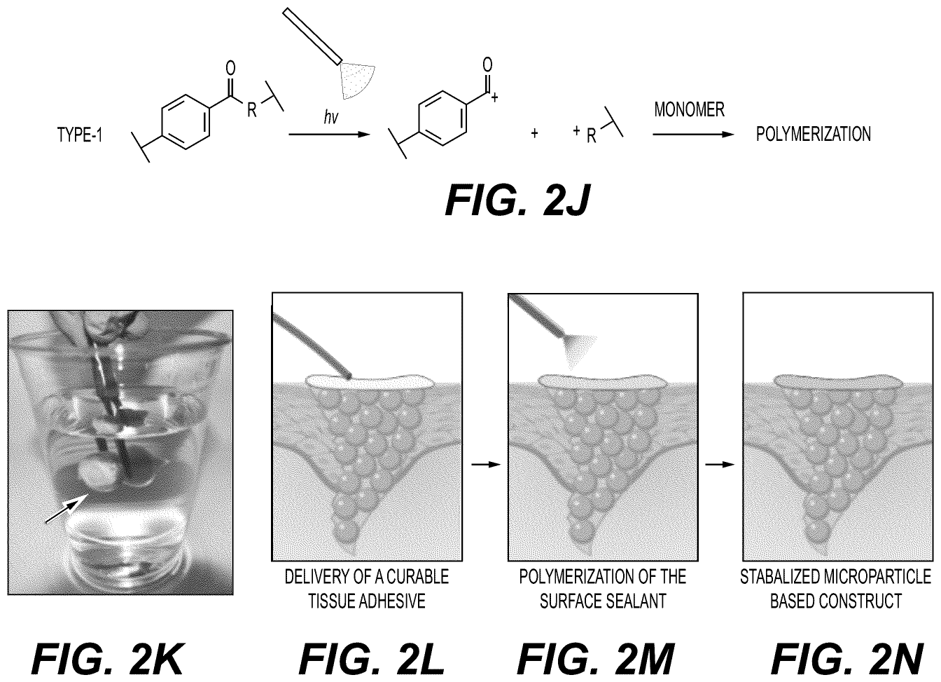

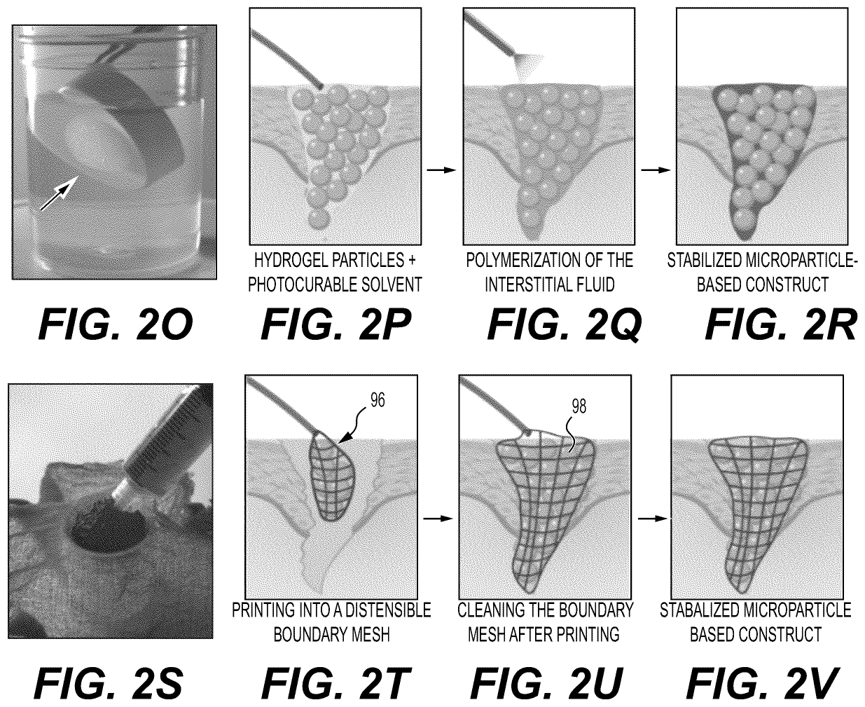

See FIGS. 2A-2I, showing how un-jammed particles (FIG. 2A, 2D) in a fluid become jammed as volume fraction increases (FIG. 2B, 2E), to become more solid, then as the volume fraction increases further, a solid structure with minimal space between particles (FIG. 2C, 2F). This process is used as the basis for additive manufacturing of microgel based constructs (FIG. 2G), as it is delivered into a tissue area in need of filling (FIG. 2H) wherein it is formed into a solid structure (FIG. 2I).

Conventional precursor solutions of viscoelastic hydrogels contain polymers that will interlock upon gelation to form a dense matrix of entangled polymer chains. The void space between neighboring polymers is on the length-scale of the original polymer, typically, nanoscale, which limits the rate of molecular diffusion and convectional fluid flow of nutrients and soluble signaling mediators through the hydrogel. Methods have been developed to create pre-formed hydrogel scaffolds with micron-sized pores that can be compressed for injection, However, these cannot conform to the shape of a wound, and generate an expansion force upon release from the syringe, which may not be suitable in confined spaces. Microparticles are formed into a viscoelastic state by applying forces to decrease the distance between the microparticles. This also leads to an alteration in the surface structure, creating a non-uniform non-curvilinear surface. wherein the bulking agent is applied with force applied during filtration, preferably by gravity, gravity-driven filtration, gravity plus additional pressure, and pressure-driven filtration, with pressures between 0.5 and 3 PSI.

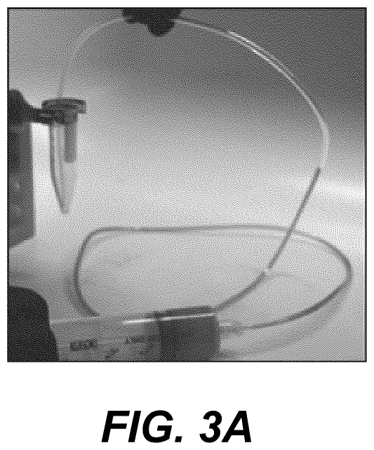

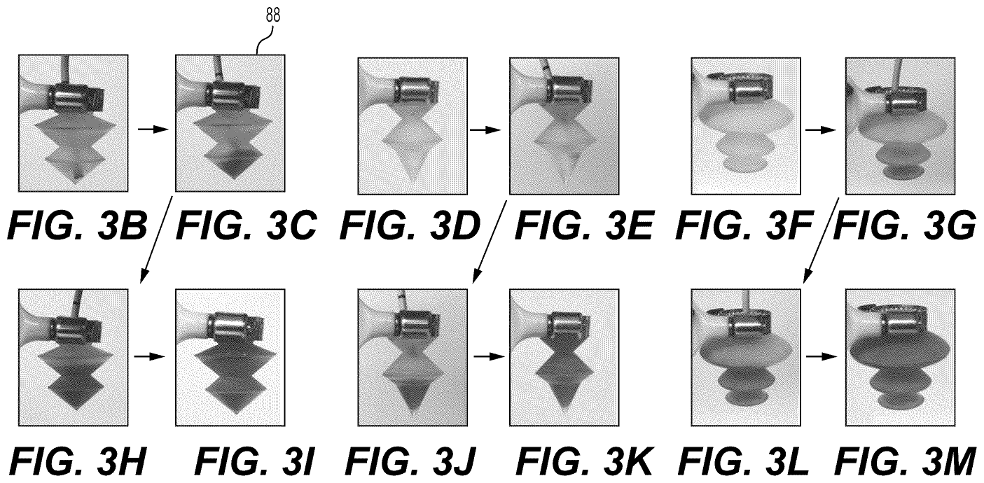

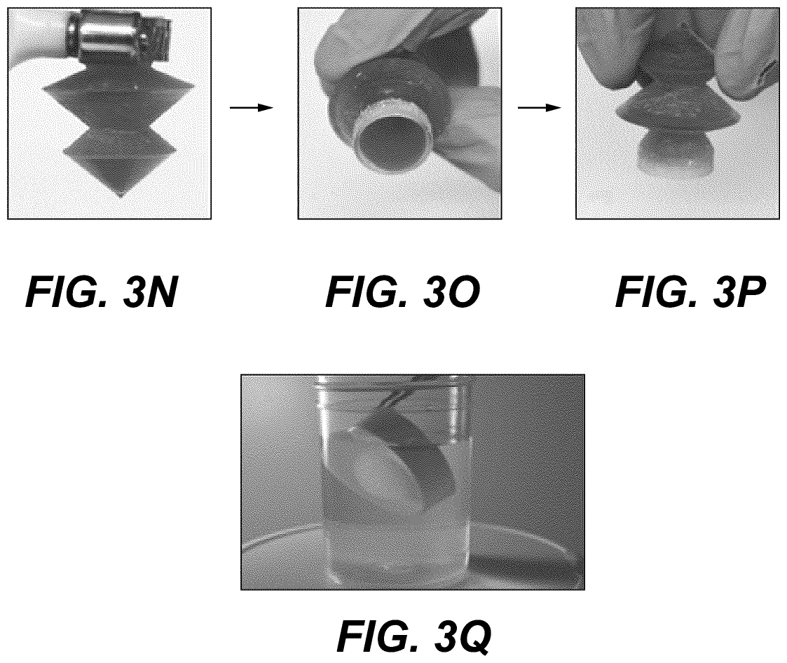

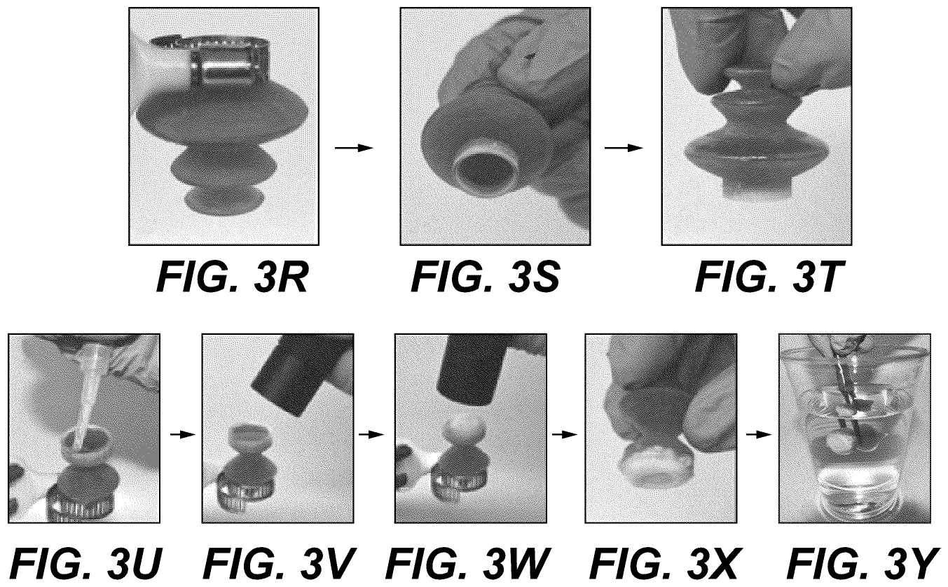

In one embodiment, microgel bioinks are composed of biocompatible alginate suspended in an isotonic buffered saline solution (fluid phase). Since the alginate microparticles are soft and deformable, their volume fraction can be increased beyond the random packing limit to form densely packed suspensions. The transition from viscous, fluid-like behavior (in the dilute limit) to complex viscoelastic behavior arises due to “jamming”. This physical transition is shown in FIG. 3A, where a dilute suspension behaves much like water, while a jammed suspension demonstrates elastic behavior with indefinite maintenance of its shape under the force of gravity. In a process known as yielding, the jammed microgels can rapidly and reversibly switch between fluid-like and solid-like properties depending on the applied shear (FIG. 3B). Below the yield stress, the jammed microgel suspension behaves elastically and maintains its bulk shape under applied forces. Above the yield stress, the suspension fluidizes leading to macroscopic flow. When the applied shear is removed, the suspension quickly recovers its bulk elasticity (FIG. 3B). The reversible liquid-to-solid behavior enables microgel printing through a narrow catheter, followed by immediate in situ stability after printing (FIG. 3C). Another critical property of the jammed microgel material is a phenomenon known as shear thinning (FIG. 3B). While Newtonian fluids display a constant viscosity, microgels exhibit a profound decrease in viscosity as the shear rate is increased. This decrease in viscosity dramatically reduces the material's resistance to flow, which facilitates the delivery of large material volumes through the narrow catheters used in clinical practice (FIG. 3C).

Alginate microgel inks produced by a droplet-based technique were able to pass through long, thin catheters (FIG. 3A). This bioink is capable of rapidly filling of arbitrary geometries (FIGS. 3B-3E), yet retains its shape after printing in arbitrary x-y-z orientations. The alginate solution was extruded through a syringe into a gelation bath containing calcium chloride. Alginate can be ionically crosslinked by a polyvalent cation such as Ca2+, Sr2+, or Ba2+ to form hydrogels. Factors affecting microparticles size and density include the alginate concentration, the volume flow rate, and the size of the syringe nozzle/needle, as well as the air flow distance and angle. Microparticles ranged in size from less than 100 to 1000 microns and were closely packed. There is variable stiffness as a function of alginate concentration from 0.5% to 2% alginate and the microparticles can be swollen in DMEM mammalian cell culture media or shrunk n calcium chloride.

When hydrogel microparticles are in a viscoelastic state, the interstitial space among the packed particles typically forms a three-dimensional, inter-connected, porous network through which cells may freely migrate and mass transport occurs. The size-scale of their pores is proportional to the size-scale of the hydrogel microparticles from which they are formed. Therefore, a micron-sized particle assembly produces micron-sized pores, which is optimal considering the micron size of most cells.

Microparticles are formed into a viscoelastic state by applying forces to decrease the distance between the microparticles. This also leads to an alteration in the surface structure, creating a non-uniform non-curvilinear surface. wherein the bulking agent is applied with force applied during filtration, preferably by gravity, gravity-driven filtration, gravity plus additional pressure, and pressure-driven filtration, with pressures between 0.5 and 3 PSI.

For example, an alginate solution is extruded through a syringe into a gelation bath containing calcium chloride. Alginate can be ionically crosslinked by a polyvalent cation such as Ca2+, Sr2+, or Ba2+ to form hydrogels. Factors affecting microparticles size and density include the alginate concentration, the volume flow rate, and the size of the syringe nozzle/needle, as well as the air flow distance and angle. Microparticles range in size from less than 100 to 1000 microns and are closely packed. There is variable stiffness as a function of alginate concentration from 0.5% to 2% alginate and that one can swell microparticles in DMEM mammalian cell culture media or shrink them in calcium chloride.

Granular hydrogels can be prepared from microgels through gravity-, pressure-, or vacuum-driven filtration, centrifugation, shear-jamming, osmotic or hydrostatic pressure gradients, or capillary wicking. The softness of the microgels enables particle deformation, faceting, and deswelling so volume fractions much greater than close packing can be achieved. The solid phase and fluid phase characteristics of the material, along with the particular jamming method and user-defined parameters, determine the rate of jamming and the maximal volume fraction that can be feasibly obtained. Both heuristic methods (measurements of solid-phase and fluid-phase volumes) and more exact methods (fluorescent labeling and 3D confocal imaging) can be used to estimate the volume fraction of the viscoelastic granular hydrogel. The user-defined customization of the solid-phase gel building blocks, fluid-phase interstitial fluid, and aggregate viscoelastic suspension volume fraction enable the attainment of diverse granular hydrogel formulations with widely-varying viscoelastic properties, yielding onsets, and shear-thinning profiles.

These techniques are compatible with mechanical, pneumatic, or manual extrusion approaches. Mechanical approaches can be as simple as loading a syringe into a syringe pump or other mechanical device, using a defined plunger displacement rate to drive material extrusion. Pneumatic approaches include using a pressure line attached to a cartridge and application of programmed pressure to drive material extrusion. Manual approaches include using manually applied to the syringe plunger to drive material extrusion. Magnetically-controlled/user-defined patterning of material can be used. The position of a ferromagnetic printhead can be manipulated via application of a magnetic field, thereby eliminating the requirement for direct contact. A human user can control the positioning of the magnet, thereby controlling the position of the printhead. This can be used to shift the entire position of the catheter or shift the position of the catheter nozzle during extrusion, thereby enabling arbitrary spatial patterning.

Deposition of discrete volumes of material in user-defined locations. One can optimize phase separation for fluid-like filling and solid-like stability. The separation of the interstitial fluid-phase and gel solid-phase can change the material properties. At the start of a print, phase separation will lead to the extrusion of material with a lower solid volume fraction. Higher fluid volume fraction may enable smooth, fluid-like filling of distal trabeculations, crevasses etc.; at the end of a print. The remaining material will be at a higher solid volume fraction/lower fluid volume fraction, which can provide much greater mechanical stability/stiffness in order to ensure the structural integrity of the deposited scaffold (which will equilibrate to the pre-delivery material volume fraction).

The properties of the gel microparticles in situ can be manipulated. To modify the properties after delivery of the material, the interstitial fluid can be withdrawn to further solidify the microgels in situ, for example, by application of negative pressure with a semi-permeable membrane to prevent gel microparticle passage) or additional fluid can be delivered to decrease the volume fraction of the microgels in situ. To modify gel microparticle properties: after delivery of the material, the interstitial fluid could be withdrawn and then replaced by a different fluid with different chemical properties (e.g. ions, salts) to induce the in situ contraction or expansion of the microgels.

C. Crosslinking Agents, Sealing and Barrier Materials

Hydrogels can be ionically or covalently crosslinked in the form of microparticles. The microparticles can also be ionically or covalently crosslinked to form a more permanent solid microparticle material.

While microgel bioinks are engineered to exhibit a solid-like response upon printing, secondary stabilization is preferred to ensure their long-term structural stability in dynamic, fluid-filled intracorporal environments. Therefore the “neck” of the defect is typically sealed with a photo-activated adhesive and/or the interstitial fluid phase of the ink functionalized to enable photopolymerization after infilling the defect site.

In the preferred embodiment, the hydrogels are ionically crosslinked to form particles. Hydrogels are typically ionically crosslinked using an agent such as a divalent cation such as calcium.

Chemical or permanent hydrogels are formed by covalent crosslinking of polymers. One common way to create a covalently crosslinked network is to polymerize end-functionalized macromers. Hydrogels are crosslinked with many compounds such as glutaraldehyde. Some other crosslinking compounds are formaldehyde, epoxy compounds, and dialdehyde. The type and degree of crosslinking influences many of the resulting properties, like swelling properties, elastic modulus and transport of molecules

Microparticles can also be crosslinked to make the material firmer and less likely to dissolve. In most cases a chemical crosslinker is used to form covalent bonds between the microparticles. For example, alginate may be ionically bound using calcium or barium, then crosslinked with a polyamino acid to form a stronger membrane surface.

Preferred methods for crosslinking are ionic (Ca2+) gelation of the microgels (alginate) and photoinitiated (405 nm UV) gelation of the functionalized fluid phase (methacrylated gelatin+LAP).

Other methods to crosslink the microgels include direct microgel crosslinking (physical methods such as hydrophobic interactions, electrostatic interactions, guest-host interactions, hydrogen-bonding, biotin-streptavidin; chemical methods like enzymatic catalysis, photo-initiated radical polymerization, click chemistry, non-enzymatic amidation), gel-mediated crosslinking (functionalized gels mixed with reactive polymers to form bulk), physical gel entrapment (gels are trapped inside another physically or chemically gelled network), as well as cellular interlinking in the interstitial space.

Photopolymerization has several advantages over conventional polymerization techniques: better spatial and temporal control of polymerization, fast curing rates (less than one second at physiological temperatures), and minimal heat production. Furthermore, photopolymerization enables the fabrication of complex geometries with both spatial and temporal control over the polymerization process.

See, for example, Farjami, T., & Madadlou, A. (2017). Fabrication methods of biopolymeric microgels and microgel-based hydrogels. Food Hydrocolloids, 62, 262-272. Feng, Q., Li, D., Li, Q., Cao, X., & Dong, H. (2022). Microgel assembly: Fabrication, characteristics and application in tissue engineering and regenerative medicine. Bioactive materials, 9, 105-119.

D. Cells, Therapeutic, Prophylactic and Diagnostic/Imaging Agents

The hydrogel microparticles can be used for delivery of therapeutic, prophylactic and/or diagnostic agents, including not just drugs but cells and other biologicals, either encapsulated in the microparticles, suspended with the microparticles, or both. For example, alginate can be ionically cross-linked with divalent cations, in water, at room temperature, to form a hydrogel matrix. See, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

Cells can be obtained directed from a donor, from cell culture of cells from a donor, or from established cell culture lines. In the preferred embodiments, cells are obtained directly from a donor, washed and implanted directly in combination with the polymeric material. The cells are cultured using techniques known to those skilled in the art of tissue culture. In the preferred embodiment, the cells are autologous, i.e., derived from the individual into which the cells are to be transplanted, but may be allogeneic or heterologous. The polymeric matrix can be combined with humoral factors to promote cell transplantation and engraftment. For example, the polymeric matrix can be combined with angiogenic factors, antibiotics, anti-inflammatories, growth factors, compounds which induce differentiation, and other factors which are known to those skilled in the art of cell culture. Cells may be pluripotent, multipotent, differentiated, genetically engineered, autologous, allogeneic, or from cell culture. Representative cell types include fibroblast, tissue cells, endothelial cells, and combinations thereof.

Agents may be proteins, peptides, carbohydrates, polysaccharides, nucleic acid molecules, or organic molecules. The preferred materials to be incorporated are drugs and imaging agents. Therapeutic agents include antibiotics, antivirals, anti-cancer (referred to herein as “chemotherapeutics”, antibodies and bioactive fragments thereof (including humanized, single chain, and chimeric antibodies), antigen and vaccine formulations, peptide drugs, anti-inflammatories, oligonucleotide drugs (including DNA, RNAs, antisense, aptamers, ribozymes, external guide sequences for ribonuclease P, and triplex forming agents).

Representative classes of diagnostic materials include paramagnetic molecules, fluorescent compounds, magnetic molecules, and radionuclides. Exemplary materials include, but are not limited to, metal oxides, such as iron oxide, metallic particles, such as gold particles, etc. Biomarkers can also be conjugated to the surface for diagnostic applications.

### III. Catheter Delivery Devices

Hydrogel microparticles, even in large volumes, can be extruded through catheters of arbitrary length, diameter, and tortuosity, using methods and device compatible with existing minimally invasive routes to target tissues e.g. percutaneous, keyhole, and are compatible with mechanical, pneumatic, or manual extrusion approaches. Catheters can be guided to remote sites using bending/rotation/pull wires. An advantage of administration with a catheter is that the same device can be used to administer materials to multiple sites, then to be modified, for example, for example, by photo-crosslinking using a fiber optic light in the catheter.

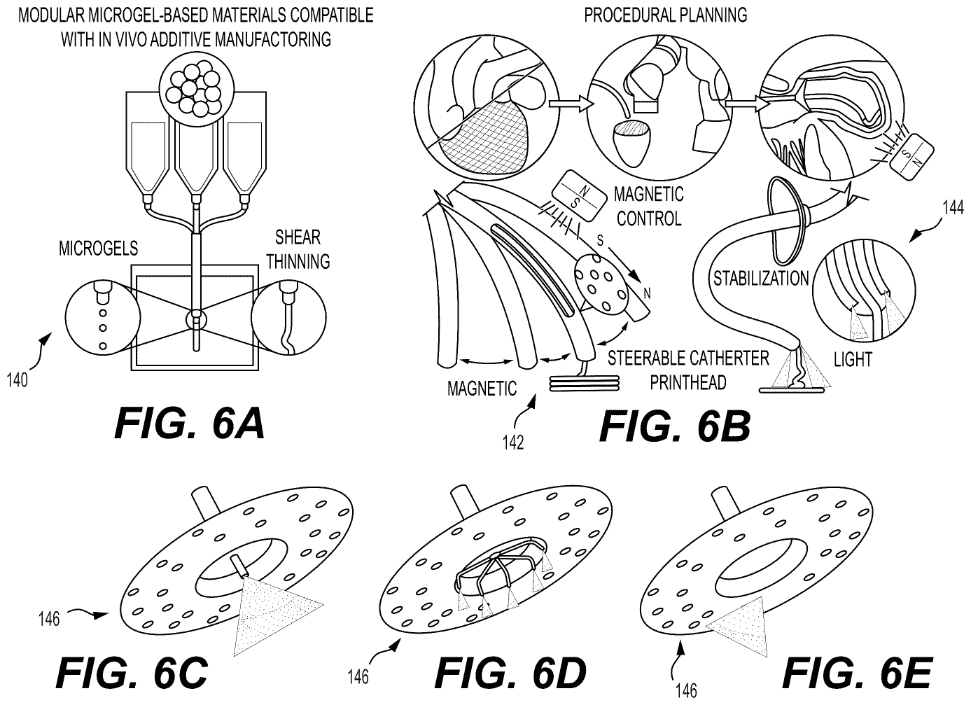

FIGS. 6A-6C are schematics of a modular system for delivery of microgel-based materials compatible with in vivo bioprinting, where the mixture is optimized using a mixture of microgel size, chemistry and shear-thinning properties to produce implants with desired properties (FIG. 6A), which can be delivered using a magnetically controlled steerable catheter to direct the materials then to solidify them, for example, by photopolymerization (FIG. 6B), where the catheter includes a print head that can provide light in a variety of patterns, diameters, and intensity to control crosslinking (FIG. 6C).

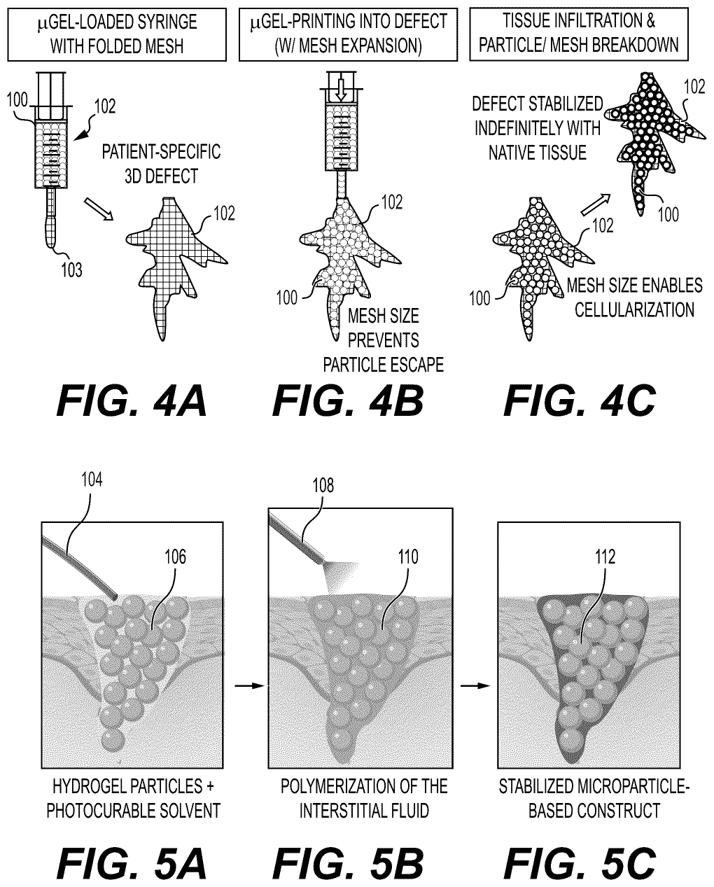

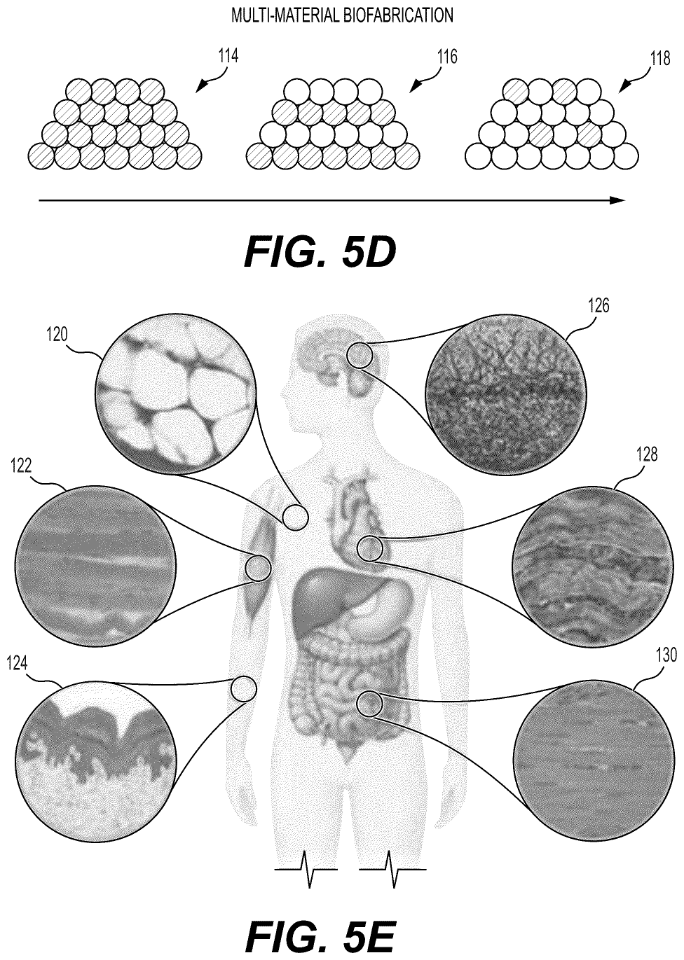

Studies demonstrate that the methods of applying these materials to a site, including using three-dimensional printing or reversible additive manufacturing, are flexible and omnidirectional, with comparable results even if oriented upside down or sideway, and that the resulting materials have a highly robust mechanical profile, maintaining properties even after months of demanding extrusion testing. See FIGS. 3A-3D. Materials may be administered with arbitrary volumes and/or geometry (planar, curved, convexities/concavities, trabeculations). Materials can be administered with a foldable scaffold. See FIGS. 4A-4C. Materials can be homogeneous, layered with different compositions, sizes, crosslinking, or density for use in specific applications where the defect to be treated is not homogenous. See FIGS. 5A-5E.

The materials can be administered using magnetically-controlled/user-defined patterning of material, be removed post administration if needed, and modified in situ. As shown in FIG. 6A, the microgel feed to the catheter can be selected to deliver materials that are optimized for the tissue to be treated, or varied during delivery as desired.

Use of an externally controlled catheter (directed using an external magnet) having a 3D printing head/photo optic fibers for crosslinking and/or sealing the microgels provides further flexibility in design and delivery. The optical fibers can be varied in number, diameter, and directionality to provide further processing options. See FIGS. 6A-6C.

The transcatheter bioprinter is able to (1) maneuver to the site of the tissue defect with enhanced surgical dexterity for complex or asymmetric defects, (2) isolate the tissue defect from the surrounding environment to create a clear, fluid-free printing space, (3) stabilize and orient the printhead in 3D space within the dynamic intracardiac environment, (4) deliver the bioink with controlled spatial resolution, and (5) deliver the appropriate light stimulus for polymerization of the bioink all in a minimally invasive manner that can be easily performed by a medical practitioner.

In one embodiment, ferromagnetic soft robotic technology is utilized to fabricate a soft robotic printhead that can be guided by an external magnet to achieve spatially controlled delivery and photopolymerization of a bioink in vivo.

The catheter includes a lumen for bioink delivery, a lumen for vacuum suction and defect emptying, and an optical fiber for delivering light to induce photopolymerization, either UV or visible wavelengths (for example, 405 nm wavelength can be used with LAP or 400-450 nm for ruthenium photoinitiator). The bioink delivery lumen (material and diameter) and printhead (diameter and shape) are optimized to minimize applied shear pressure required to successfully extrude the ink through the catheter in a controlled manner with millimeter resolution and to maximize light delivery for efficient photopolymerization.

A vacuum-based tissue gripper can be incorporated to be deployed at the end of the catheter for defect isolation and printhead stability during material deposition. The vacuum-based tissue gripper, preferably formed of silicone, combines vacuum suction and, in a preferred embodiment, a biologically inspired octopus design, for wet-tolerant tissue adhesion. Adhesion forces for various gripper architectures are optimized and controllable for different preloads (0-30 kPa) and different surface conditions (dry, moist, under water) for optimal tissue gripping and maintenance of adhesion. In one embodiment, for defect isolation and printhead, a self-expanding peri-defect ring is exposed and connected to a vacuum source. This creates a ring of suction around the defect site to connect the catheter to wet and dynamic surfaces. Following the initial attachment, an inner catheter is advanced and connected to a different vacuum source. This allows for removal of any fluid or emboli found within the defect. After evacuating the defect site, the second vacuum is turned off while the first vacuum remains on, creating a sheltered, fluid-free 3D workspace for controlled bioprinting with sustained separation from the vasculature. After achieving this isolated environment, the printhead can be advanced to pattern the microgel ink in the open volume. Using this approach, a catheter can be stably attached to soft and wet surfaces, a fluid-filled defect site can be evacuated to provide a clean workspace for in vivo printing, and (3) a microgel ink can be printed into the isolated space to fill the defect.

The entire catheter system should be compatible with existing introducer sheaths, endoscopes, cystoscopes and trackable over intravascular guidewires, depending on the specific application. Combined with existing imaging modalities, these methods can be used to deliver the catheter-based bioprinter to the desired target location. Integrating steerable technologies into catheters enables the operator to vary the distal shape of the catheter and select the desired direction of motion. The most common steerable catheters make use of four main actuation mechanisms: pull-wire, smart-material-actuated, hydraulic drives, and magnetic. Pull-wire catheters rely on a tendon-based continuum system, in which a super-elastic nitinol catheter is steered by actuating tendons that are terminated at the catheter tip. Smart-material actuated catheters include shape memory alloys (SMA) whose elastic properties vary with temperature, allowing bending and deflection of the catheter tip through heating and cooling of the SMA actuator. These actuators have not been widely accepted in commercial systems due to potential dangers of overheating. Hydraulically actuated catheters use a series of bellowed segments that can bend in a single plane by injection a fluid into the bellows. While the hydraulic approach forgoes the need for electrical communication or driving circuitry, it is difficult to continuously control the bending of the individual segments, thus this method has failed to enter mainstream commercial technologies. Magnetic steering relies on specialized catheters and guidewires that have magnetic components at tip and are controlled by the magnetic field of an external permanent magnet. Due to the soft tip of magnetic catheters, they are safer than pull-wire and smart material-actuated catheters, which require a certain stiffness to maintain catheter shape.

A magnetic catheter navigation system, which offers improved accessibility to the site of interest, improved catheter stability in operation, and decreased patient risk, is therefore preferred for spatially controlled, patient-specific therapy to the LAA in a minimally invasive manner.

A. For Delivery of Hydrogel Microparticles

The target application informs the catheter choice. For example, catheters for cerebral aneurysm coiling may be on the order of approximately 1.6 Fr and less than 150 cm, while catheters for LAA occlusion may be on the order of approximately 10-14 Fr and approximately 90-150 cm.

Catheters or syringes for extrusion of the microparticles can be used to create a physical block, to close off an area or seal a leakage. Catheters are commercially available. Syringes and material cartridges may range from 1 mL to 25 mL. Catheter outer diameters may range from approximately 1.5-2 Fr to 14 Fr, and the lengths may range from 10 cm to 150 cm. Beyond catheters, needles and injection cannulas can also be used for material delivery, with inner diameters ranging from 0.1 to 3 mm. Catheter tips and injection nozzles may be straight or tapered and different types of inner coatings may be applied (e.g. deposition of hydrophobic layers). The method of delivery can be manual injection, mechanical extrusion, or pneumatic extrusion.

Gel microparticle suspension undergoing laser-prescribed phase separation, showing viscoelastic microparticles and solid viscoelastic microparticles under increasing pressures. Viscoelastic microparticles are placed in a syringe to form granular hydrogels for extrusion printing. Microparticles are converted to viscoelastic microparticles by extrusion through a syringe and then loaded into a catheter or syringe for application to a site to form a solid three-dimensional hydrogel structure. One can extrude viscoelastic microparticles using a catheter based delivery system which can be filled into complex three-dimensional geometries and volumes. FIGS. 3A-3D shown that the viscoelastic microparticles can be formed into filaments (FIG. 3A) or filled into the complex three-dimensional geometries from any orientation: top to bottom, (FIG. 3B); sideways (FIG. 3C). Granular hydrogels display instant solid-like stability and can be sealed or bulk-polymerized (FIG. 3D).

B. For Sealing and Barrier Formation

Microgels are micron-sized microparticles composed of hydrogels. They can be generated using a variety of fabrication methods including emulsification using ultrasonication, mechanical agitation or high-pressure homogenization, atomization, extrusion through a syringe or nozzle, micromolding, and molecular self-association. The solidified hydrogel microparticles provide immediate, solid-like stability.

Various ionic crosslinking methods for gelation of charged polymers are known, including external gelation via crosslinkers dissolved or dispersed in the oil phase, internal gelation methods using crosslinkers added to the dispersed phase in their non-active forms, such as chelating agents, photo-acid generators, sparingly soluble or slowly hydrolyzing compounds, and methods involving competitive ligand exchange, rapid mixing of polymer and crosslinking streams, and merging polymer and crosslinker droplets. Covalent crosslinking methods using enzymatic oxidation of modified biopolymers, photo-polymerization of crosslinkable monomers or polymers, and thiol-ene “click” reactions are also useful, as well as the methods based on sol-gel transitions of stimuli responsive polymers triggered by Ph or temperature change. In ‘physical’ hydrogels, molecular entanglements and/or secondary forces such as ionic, H-bonding or hydrophobic forces play the main role in the network formation.

Physical gels are reversible and can be disintegrated by changing environmental conditions, such as Ph, temperature, and ionic strength of the solution. Typical physical hydrogels, such as alginate, carboxymethyl cellulose and chitosan, are prepared by ionotropic gelation with oppositely charged divalent ions. In ‘chemical’ gels, polymer chains are permanently connected by covalent bonds. Chemical gels can be prepared in two different ways: free radical polymerization of low molecular weight hydrophilic monomers and polymerization of polymers. Free-radical polymerization often results in a significant level of residual monomers, and therefore, hydrogels preferably are be purified to re move unreacted monomers, which are often harmful.

The mechanical properties of ionically crosslinked natural polymers, such as elastic modulus, and swelling ratio, may be unstable due to potential loss of crosslinking ions. However, functional groups (e.g., —OH, —COOH, and —NH₂) of natural polymers can be chemically modified to allow for covalent crosslinking. For example, phenol containing molecules such as tyrosine and tyramine can be conjugated to alginate via carbodiimide chemistry or periodate chemistry. The alginate-tyramine conjugates can be crosslinked via horseradish peroxidase (HRP)-catalyzed oxidative coupling of phenol moieties in the presence of hydrogen peroxide (H₂O₂). Gel networks composed of covalently cross-linked polymer chains have better mechanical properties and greater chemical and thermal stability compared to ionically crosslinked polymer networks Hyperbranched polyglycerol (Hpg) and polyethylene glycol (PEG) can be functionalized with acrylate groups and undergo free radical co-polymerisation within cell-laden droplets upon UV irradiation in the presence of a photoinitiator. Another example of polymer-polymer crosslinking by click chemistry is the reaction between azide-functionalized poly(N-(2-hydroxy-ypropyl)-methacrylamide) (PHPMA) chains and cyclooctyne-functionalized poly(N-iso-propylacrylamide) (PNIPAAm) and poly(ethylene glycol) (PEG) chains. For photopolymerisation, droplets composed of a mixture of functional monomers and photoinitiators are exposed to UV or visible light to initiate free-radical polymerisation. Formation of biocompatible hydrogels re-quires the use of cytocompatible photoinitiators, such as IRGACURE® 2959, 1173, 819, and 651, riboflavin phosphate, camphorquinone, and eosin Y. Visible light photoinitiation is advantageous for encapsulation of biological materials since UV radiation can cause DNA damage and accelerate tissue aging and cancer onset. Blue light photo-initiators that can be used are camphorquinone, eosin Y, and riboflavin. Common gel microparticles produced by monomer crosslinking with UV light are poly(N-isopropylacrylamide) (PNIPAAm) and polyacrylamide (PAAm).

Water soluble pre-polymers modified by introduction of cross-linkable molecules can be used instead of monomers. The examples of such modified polymers used for microfluidic production of gel microparticles are dextran-hydroxyethyl methacrylate (dextran-HEMA), gelatin-methacryloyl (GelMA), poly(N-isopropylacrylamide-dime-thylmaleimide), (P(NIPAAm-DMMI)), poly(ethylene glycol diacrylate) (PEGDA), poly(ethylene glycol methyl ether acrylate) (PEGMA), poly(ethylene glycol) norbornene (PEG-NB), and 6-armed acrylated PEG. Natural polymer conjugated with photopolymerizable groups are attractive alternatives to synthetic hydrogels, because they can combine light polymerizable groups with inherent cell adhesion properties, due to the presence of natural cell-binding motifs, and excellent biodegradability, due to the presence of enzyme-sensitive links. An examples is gelatin methacryloyl (GelMA), which is synthesized through the reaction between gelatin and methacrylic anhydride. The conjugation of the methacryloyl moieties occurs mainly on primary amine groups of lysine and hydroxylysine residues. Thermo-responsive hydrogels can be divided into two groups: upper critical solution temperature (UCST) hydrogels and lower critical solution temperature (LCST) hydrogels. UCST hydrogels such as gelatin and agarose are formed by cooling polymer solution to below a UCST.

Surface sealing prevents gel microparticle scaffold from interfacing with fluid. Bulk polymerization can be used to lock the entire scaffold in place. Alternatively, printing into a distensible boundary mesh can be used to prevent gel microparticle migration or escape. The compliant boundary allows particles to occupy the defect volume but prevent the particles from escaping the net. This additional layer of security could be particularly useful in high-risk applications like intracardiac or intravascular defect closure where embolization must be avoided. Materials have a two to three year shelf life, even at room temperature.

Surface sealing can be performed by chemical crosslinking. Reactants can be premixed and then deposited on the surface (where curing will take place over time) or functionalized precursors can be deposited on the surface and then photopolymerization can be used to cure the seal. Alternatively, preformed patches or adhesives could be applied to the surface.

The physical barriers will be comprised of malleable meshes that will be pre-loaded onto the catheter tip. The mesh porosity will be smaller than the particle diameter, such that particles cannot escape but cells or vasculature could infiltrate the scaffold. The mesh could be composed of either biodegradable or permanent materials (of either a natural or synthetic origin).

C. Formation of Tissue in Combination with Support Structures

FIGS. 4A-4C are schematics of the process of repairing a three-dimensional tissue defect by extruding viscoelastic microparticles 40 in combination with a surgical mesh 42 through a syringe 44, where the gel is administered with the folded mesh, the mesh size prevents particle escape but allows circulation, resulting in stabilization of the defect as cells infiltrate, using the microparticles and mesh as scaffold to form tissue to permanently repair the defect. Other mechanical barriers could be used in place of, or in addition to, the surgical mesh. The microparticles can be administered as a suspension alone or within or in abutment with a mesh to seal in the implanted microparticles.

Other support structures can be used, but clips, sutures, rings, and pins preferably should be avoided.

D. Photopolymerization Formation of Tissue Structures

FIGS. 5A-5C are schematics showing that bulk polymerization of extruded viscoelastic microparticles in a three-dimensional defect can provide long term stability. Hydrogel particles 50 and photocurable solvent are injected into the tissue defect. These are photopolymerized 52 with the interstitial fluid-microparticle mixture 54 to yield a stabilized microparticle based construct 56.

FIG. 5D shows how the microparticles may be homogeneous, layered of microparticles with different composition, or spatially heterogeneous. FIG. 5E demonstrates how that can be used to more accurately reflect tissues that are not just homogeneous cells, but mixtures of cell types, having different physical and structural properties. No other method is known to be capable of this kind of spatial and compositional complexity other than by tissue transplantation.

### IV. Methods of Tissue Repair and Barrier Formation

The technology is useful for intracardiac, gastrointestinal and gynecological tissue defect repair. In one embodiment, gel microparticles are printed into explanted tissue defects, where the gel is applied as a liquid suspension into the defect; to form a solid-like three-dimensional microspatial structure; then sealed with crosslinking and/or tissue adhesive to form a permanent structure.

The technologies described herein provide a trans-catheter 3D bioprinting platform enabling customized, biocompatible fabrication at the site of a patient's tissue defect in a minimally-invasive fashion. Bioinks composed of hydrogel microparticles can be printed in situ through a robotic catheter. The mapping of tunable input parameters (e.g., microparticle diameter and fluid phase viscosity) to desired printing outcomes (e.g., shear-thinning profile and rapid elastic recovery) produce design rules for creating bioinks for specific clinical applications. Catheter-based technologies support controlled biomaterial delivery in vivo. Essential design features include navigation to the tissue site, isolation of the defect from surrounding tissue, controlled delivery of the bioink in three dimensions, and stabilization of the bioprinted construct to ensure long-term structural integrity. These technologies provide the means for treating myriad clinical indications with varying tissue properties and anatomic location.

Beyond the variability introduced by differing types of defect, even a single defect type (e.g. atrial appendage or cerebral aneurysm) displays huge patient-to-patient variability. The morphological heterogeneity of these defects precludes the possibility of a prefabricated, one-size-fits-all solution. Traditional manufacturing techniques simply cannot match the wide range of defect volumes and geometries required for patient-specific therapy.

Congenital, acquired, and iatrogenic 3D tissue defects arise in a number of poorly accessible locations throughout the body, ranging from intracardiac and intravascular defects, to gastrointestinal and gynecological defects. Though these defects differ in etiology and prognosis, they are united by the fact that their abnormal structure leads to adverse downstream consequences for patients. As a result, physicians may pursue a variety of strategies to repair the defects to improve patient health. Current approaches for volumetric filling of defects include conventional surgical repairs, traditionally manufactured medical devices, additively manufactured constructs, and injectable biomaterial implants. The method chosen to repair these defects depends on severity of the defect, location of the defect, patient status and goals of the therapy. Therapeutic goals include prevention of clot formation, reduction of inflammation, separation of cavities, starvation of tumors, restoration of injured, surgically-modified, or adversely-remodeled tissues, and filling of voids left behind after tumor, fibroid, or cyst removal. See Table 1:

TABLE 1
Comparison chart summarizing advantageous features of the proposed technology The technology has clear advantages over
conventional surgical repairs, traditional medical devices, constructs that are 3D printed a priori or conventional
injectable biomaterials. Each have inherent limitations in procedural time, risk, scalability or patient specificity.

| | | | | Biocompatible | |
|---|---|---|---|---|---|
| | | Conventional | Traditional | Constructs | Injectable |
| Manufacture | Proposed | Surgical | Medical | Manufactured | Biomaterial |
| Method | technology | Repairs | Devices | with AM | Implants |
| Examples of | Left atrial | Surgical | Transcatheter value | Craniofacial defect | Subcutaneous |
| Clinical | appendage closure, | aneurysm closure, | repair, transcatheter | repair, bone defect | injections, |
| Application | gynecological | arteriovenous | occlusion of | repair, cartilaginous | intramyocardial |
| | defect closure | malformation | intracardiac | repair | injections |
| | | closure | defects | | |
| Invasiveness | Minimally | Highly | Minimally | Typically, highly | Minimally to highly |
| | invasive | invasive | invasive | invasive | invasive |
| | | | | implantation | |
| Patient | Highly specific with | Patient specific, | Usually come in | Highly specific | Low specificity |
| Specificity | tunable mechanical | but surgical | size ranges (e.g., | | |
| | properties and | implants/patches | occluder | | |
| | tunable geometries | often flat, and | devices/valves) | | |
| | | manually shaped | requiring multiple | | |
| | | by surgeons | devices to be in | | |
| | | | stock in hospitals | | |
| Procedural Time | Relatively | Lengthy surgeries | Can require multiple | Lengthy surgeries, | Often required |
| | | | ancillary devices | pre-procedural wait | multiple small volume |
| | | | and procedures | while implant is | injections |
| | | | | being manufactured | |
| Scalability | High - the same | Limited by | Patient | Time intensive | Scalable, but time- |
| | catheter system can | surgical operating | heterogeneity | production, | intensive for multiple |
| | be used for | room time, | precludes a | manufacturing | injections |
| | heterogeneous | surgeon | prefabricated one- | chain limited by | |
| | defect filing | availability, etc. | size-fits all device | printer availability | |
| Anesthesia | Local or none (for | General | Local to general | Often implanted | Local (subcutaneous) |
| Levels | NOTES | anesthesia | depending on type | surgically requiring | or general |
| | procedures) | | of device | general anesthesia | (intramyocardial) |
| Level of Risk to | Low risk, | High risk, longer | Low procedural | High risk due to | High risk if invasive |
| Patient | minimally invasive, | stays, possibility | risk, but implant can | surgical | implantation, risk of |
| | biopolymers that | of infection, often | lead to tissue | implantation-longer | dislodgement with |
| | are unlikely to | cardiopulmonary | erosion, | stays possibility of | endocardial injection |
| | cause tissue | bypass for cardiac | paravalvular | injection, longer | |
| | damage/cytotoxicity | devices | leakage, migration, | recovery times | |
| | | | infections, | | |
| | | | conduction block | | |
| Limitations | Risk of embolism | May be | Often can't match | Require surgical | Small volumes, lack |
| | that can be | contraindicated or | patient variability, | implantation, long | specificity and spatial |
| | mitigated by | impractical | bulky, can lead to | wait times to | control |
| | secondary | depending on | tissue | produce implant, | |
| | stabilization | defect | erosion/conduction | necessity for | |
| | | morphology and | problems | bioprinters | |
| | | location | | | |

Occlusion of the Left Atrial Appendage

Repair/Closure of Septal Defect

Patients with atrial fibrillation (AF) have a fivefold increase in the incidence of stroke with 90% of thrombi (blood clots) originating in the left atrial appendage (LAA). The LAA is a tubular out-pouching of the left atrial chamber that has complex and highly variable anatomy.

Since atrial fibrillation (AF) causes stroke, and strokes are associated with thrombus formation in the LAA, and because thrombi cause strokes by embolisation to the cerebral circulation, there is an urgent need for a solution to this problem of how to close the LAA, an ear-shaped sac extending from the left atrium, that is prone to blood stasis and subsequent blood clotting due to its narrow and long tubular connection with the atrium. In patients with atrial fibrillation, 91% of left atrial clots originate in the LAA. If these clots embolize to the cerebral vasculature, they have the potential to cause a stroke. While stroke risk can be managed with blood thinners, this therapy is not suitable for every patient due to contraindications and increased risk of bleeding.

Current non-surgical treatment is oral anticoagulation, which has serious side effects, poor compliance, a narrow therapeutic window, and a risk of bleeding.

The current interventional approach to reduce stroke risk is the implantation of a preformed, rigid occlusion device that plugs the LAA space to prevent the development and embolization of clots. However, such an off-the-shelf approach is not always amenable to the highly complex and diverse inter-patient anatomy, leading to patient exclusion or inconsistent device implantation and subsequent treatment failure. A study found that 36% of implantation procedures were incomplete, an event that could further potentiate the thrombogenic potential of the appendage. One group has explored the use of 3DP to create custom implants; however, these implants are still fabricated ex vivo in a time-intensive procedure and then require invasive surgery for implantation. There is a pressing unmet need for a consistent, minimally-invasive, patient-specific approach to LAA occlusion. Successful development of an intracardiac and intravascular manufacturing technique could also be used for other indications like paravalvular leakage, cerebral, aortic, and peripheral aneurysm, and arteriovenous malformation.

The techniques for mechanical occlusion are open heart surgery or percutaneous treatment, i.e., placement of a rigid structure to seal the opening, such as the WATCHMAN®, AMPLATZER®, and PLAATO® devices. These devices vary in diameter from 16-22 mm, however the appendage can vary in diameter from 10-40 mm and is only “round” in less than 10% of cases. As a result of their rigid structures and standard circular geometries, many leak, increasing the risk of thrombus formation, and many are oversized by 10-20% to reduce the risk of leakage and device embolization, so many of the percutaneous devices are unstable and at risk of movement.

Referring to FIGS. 6A-6B, the microparticle formulations can be used to fill and seal the appendage, using a syringe to insert the microparticles, optionally with a surgical mesh or into a surgical mesh or casing, to fill the appendage, then the appendage can be sealed, for example, by photocrosslinking or tissue adhesive. Due to the pore size between the viscoelastic microparticles, cells can infiltrate into the opening and replace the matrix with tissue, thereby permanently closing off the appendage.

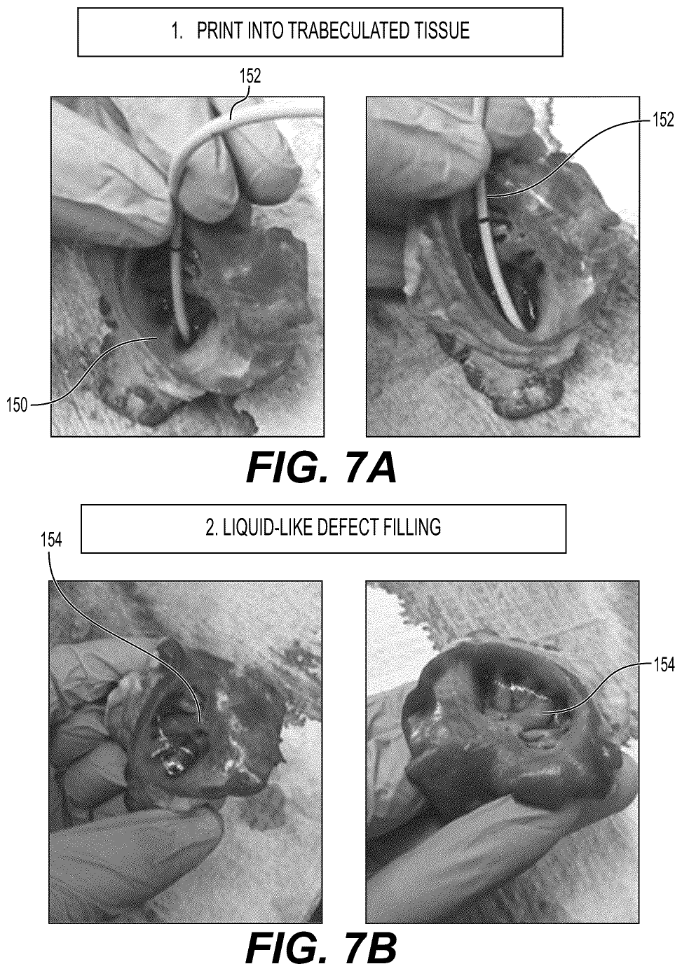

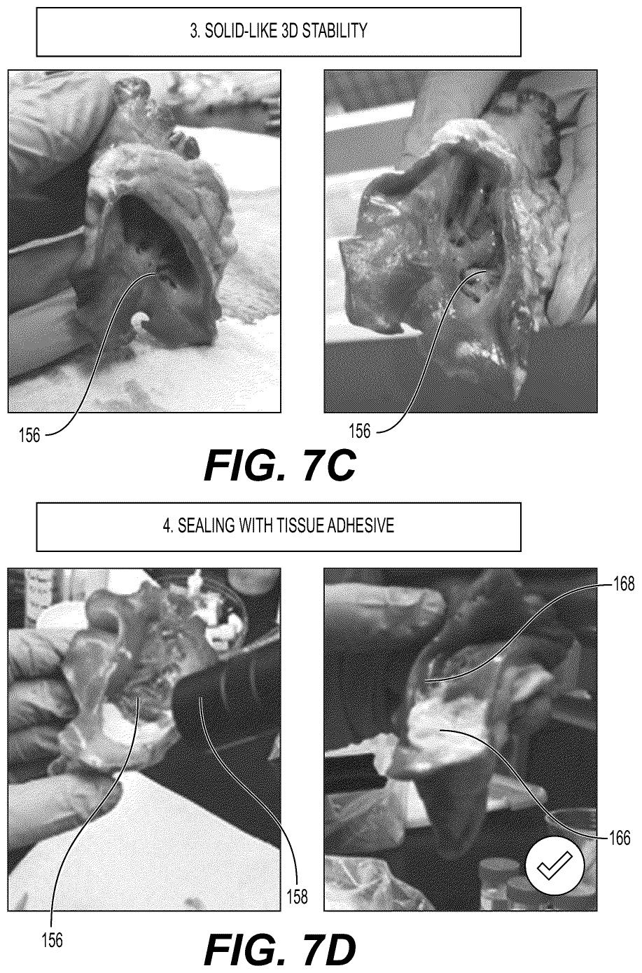

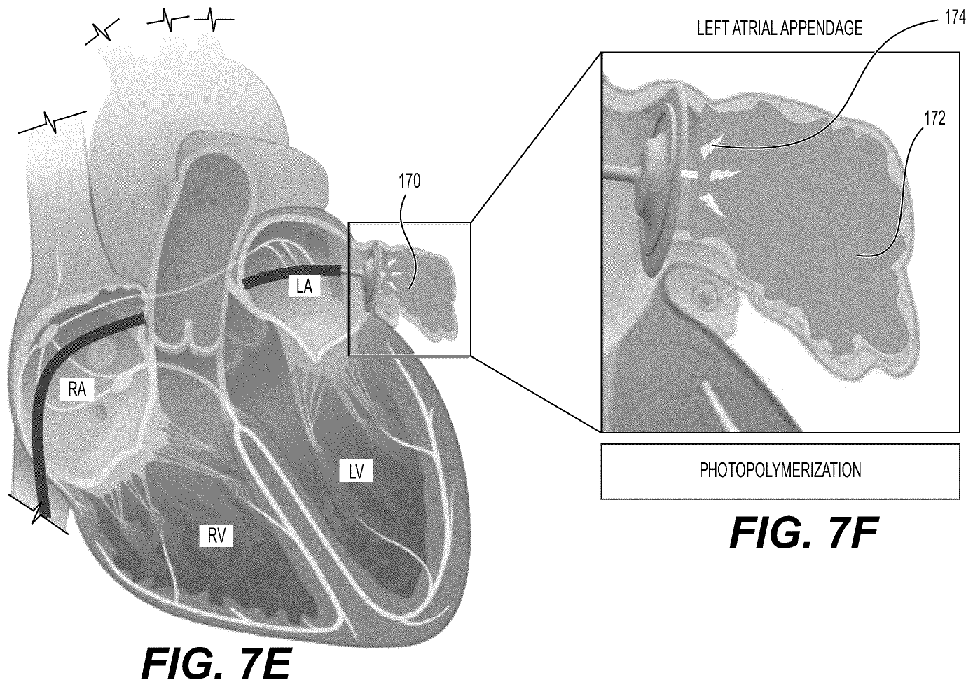

This embodiment is exemplified for the repair of a left atrial appendage or septal defect by injection of gel microparticles into the defect to form a three-dimensional structure, which is further stabilized by dispersing light through a light diffusing fiber tip to the top of the gel structure to polymerize the gel and thereby decrease the risk of embolization and increase long term occlusion stability. This process is shown in FIGS. 7A-7E, using the device of FIGS. 6A-6C.

FIGS. 7A-7B are prospective schematics of the repair of a left atrial appendage 70 by injection of gel microparticles into the defect to form a three-dimensional structure 72 (FIG. 7A) which is further stabilized by dispersing light through a light diffusing fiber tip 74 to the top of the gel structure to polymerize the gel 72 and thereby decrease the risk of embolization and increase long term occlusion stability. Gel microparticles 60 are printed into explanted tissue defects, to show that one can place a catheter 62 (as shown in FIGS. 7A-7C) at the desired site (FIG. 7A); administer the gel 60 as a liquid suspension (FIG. 7B) into the defect 64; with sufficient pressure to form a solid-like three-dimensional microspatial structure 66 (FIG. 7C); then seal 68 with tissue adhesive to form a permanent structure 70 (FIG. 7D). FIG. 7C is a schematic of the design and optimization of fiber optic for light dispersion as shown in FIG. 7B, showing a glass fiber light source (405 nm). In this example, the light source is a 600 μm glass fiber bundle 80 with a fiber tip 82, with total light intensity of W/cm2 (range 0.10 to 0.14 W/cm2) and total power of 0.10400 watts.

The deposited granular hydrogel was stable within the structure.

Repair of Torn Areas of Urethra or Vagina

From a global health perspective, there is significant need for a low-cost, non-surgical, widely applicable treatment for obstetric fistulae. Vesicovaginal fistula (VVF), the most common obstetric fistula, is an abnormal connection between the bladder and vagina that is estimated to affect over three million women worldwide. VVF occurs primarily in women in developing countries who experience obstructed labor without access to adequate obstetric care. In another example, microparticles are applied using a cystoscope into the torn areas of the urethra and vagina to form blockages to the passage of urine and fecal matter into the vagina (vesicovaginal fistula). Cystoscopes and endoscopes used for gynecological and gastroenterological purposes often incorporate light fibers for visualization, which could be coupled with a light emitting diode in the correct wavelength range at the proximal end for photopolymerization at the distal end.

VVF has a significant impact on quality of life, with women often ostracized or cast out by their communities. Surgical repair is the gold standard treatment; however, it is often unavailable for women in developing countries due to cost, geography, or a shortage of trained medical practitioners. Furthermore, if women do have surgery, repair is not always successful due to the difficultly of the procedure given complex defect shape or location. There is clear unmet need for an approach that is low-cost, easily deployed by a large cross-section of medical personnel, and widely applicable in a diverse population of patients.

The system described herein can be used for treatment or repair of vascular aneurysms (cerebral, abdominal, peripheral), vascular malformations, esophageal diverticula, deep soft tissue lesions and iatrogenic injuries, tumor resection, and emphysematous lung volumes.

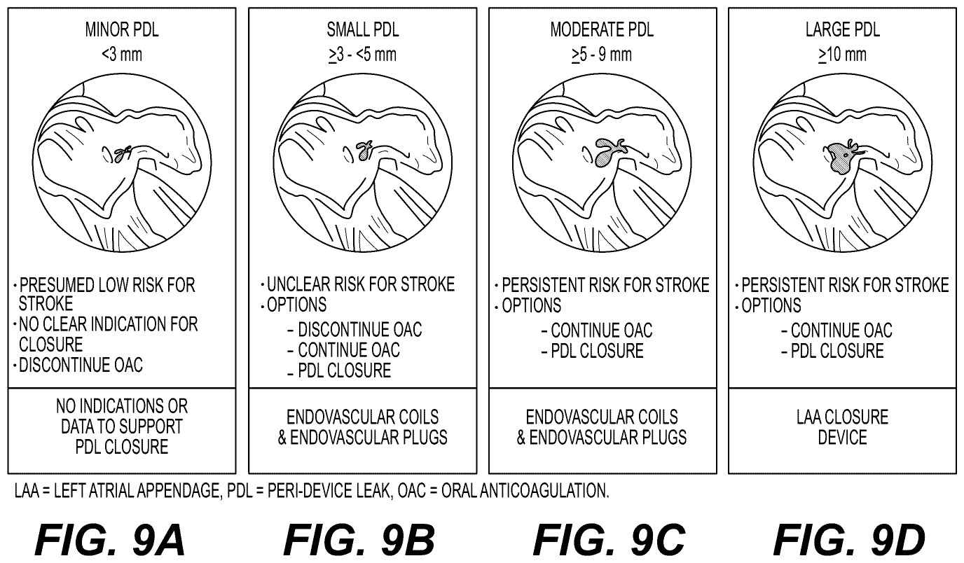

Other applications include catheter delivery of gel microparticles to form a solidified microparticle three-dimensional structure to repair peri-device leaks, where small and moderate leaks are associated with endovascular coils, endovascular plugs and large leaks is associated with an LAA closure device, or leaks associated with TAVI (percutaneous aortic valve replacement, also known as percutaneous aortic valve implantation, transcatheter aortic valve implantation or transcatheter aortic valve replacement). See FIG. 9A-9D.

Repair of Fistula

In an exemplary embodiment, a medium and a small fistulae were created in explanted porcine tissue. A catheter was advanced to the proximal portion of the fistulas and withdrawn while extruding viscoelastic microparticles into the fistula. The catheter was left in the distal portion of the fistula and microparticles extruded from there.

Adhesive was applied to seal the microparticles, thereby demonstrating adhesive delivery: The adhesive formed a watertight seal after curing. In some embodiments, adhesive can be mixed with the gel microparticles and polymerized to form an even more stabilized solid implant. Drugs can be administered with the gel microparticles, such as local anesthetic, anti-inflammatory, and/or anti-infective agents.

A fistula is an abnormal connection between two body parts, such as an organ or blood vessel and another structure. Fistulas are usually the result of an injury or surgery. Infection or inflammation can also cause a fistula to form. Fistulas may occur in many parts of the body. For example, they can form between an artery and a vein, the aorta and trachea; bile ducts and the surface of the skin (from gallbladder surgery); the cervix and vagina; the neck and throat; the space inside the skull and nasal sinus; the bowel and vagina; the colon and surface of the body, causing feces to exit through an opening other than the anus; the stomach and surface of the skin; and the uterus and peritoneal cavity (the space between the walls of the abdomen and internal organs).

These may result from complications of labor, injury, or inflammatory bowel disease, such as ulcerative colitis or Crohn disease, which can lead to fistulas between one loop of intestine and another. Injury can cause fistulas to form between arteries and veins.

Types of fistulas include: blind (open on one end only, but connects to two structures); complete (has openings both outside and inside the body); horseshoe (connects the anus to the surface of the skin after going around the rectum); and incomplete (a tube from the skin that is closed on the inside and does not connect to any internal structure).

While surgical therapy is still the main approach, it poses a risk of incontinence and poor outcomes, especially for high and complex fistulae. Newer combined techniques such as modified Seton and LIFT-plug seem relatively effective but need further study. Filling methods such as fibrin glue and fistula plug have poor outcomes with high variability. There is a high long-term recurrence rate for filling methods and fistula plug. Other problems include expulsion of fibrin clot for fibrin glue and incomplete obliteration of tract for fibrin glue, and continued infection.

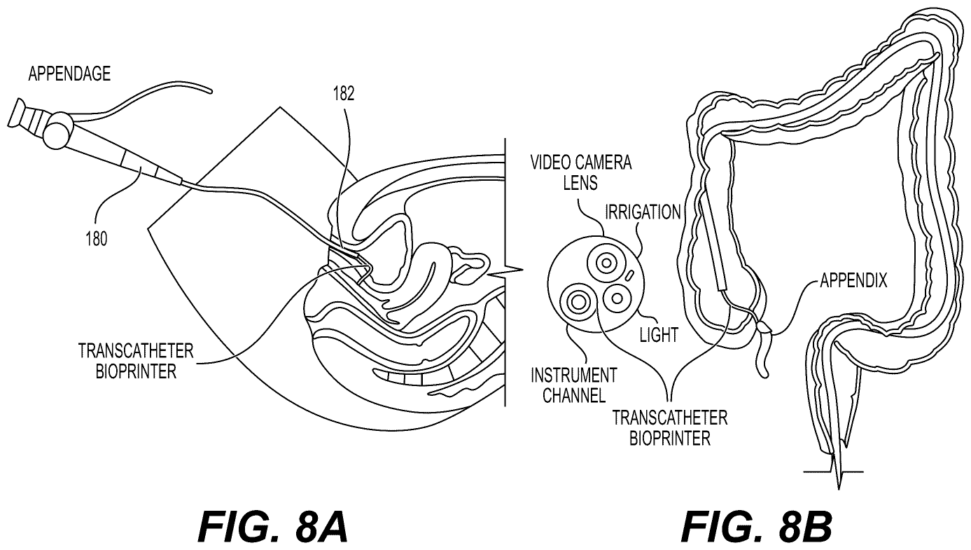

Vesicovaginal fistula (VVF) is an abnormal connection between the bladder and the vagina through which urine leaks continuously. It is caused by prolonged, obstructed labor. Connection from anus to external requires plugging. FIGS. 8A and 8B are cross-sectional schematics of the insertion of gel microparticles using a cystoscope int the torn areas of the urethra and vagina (FIG. 8A) to form blockages to the passage of urine and fecal matter into the vagina (FIG. 8B).

As shown in FIG. 8A, a cystoscope 100 is used to insert the gel microparticles into the fistula 102, then the matrix can be sealed or crosslinked for greater stability. The same process can be used to fill anal fistulas or repair anal abscesses.

Deposition of Drug Depot

The technology can be used with existing catheters or colonoscopes to construct patient-specific drug depots in the appendix, as shown in FIG. 8B. Here the unmodified particles from the standard ink are replaced with drug loaded microgels, which would then act as discrete building blocks for bottom-up fabrication of personalized drug-delivery constructs in situ.

One application is the treatment of acute appendicitis. The appendix is a small, sock-like structure that extends off of the cecum in the right lower quadrant of the abdomen. Like other anatomic structures, the appendix displays a wide range of shapes, volumes, and orientations. The standard response for appendicitis is appendectomy, but recently antibiotic therapy has emerged as a potential non-surgical treatment. In antibiotic-based therapies, patients are dosed intravenously and then orally for up to 15 days with a combination therapy containing multiple antibiotics. The extended systemic dosing schedule and incorporation of multiple drugs motivates a local, patient-specific solution, which can be fabricated in vivo in a minimally-invasive way using this technology platform.

This catheter based drug depot system eliminate patient non-compliance and reduces side effects by providing long-term, localized delivery. These drug-loaded implants have demonstrated efficacy in applications as varied as women's health, chemotherapy, and chronic pain management, but the customization of these devices is limited by available manufacturing methods. This technology obviates the problems with available devices.

Repair of Peri-Device Leaks

The compositions can be used to repair leaks in previously implanted devices, whether for atrial appendage blocking devices as discussed above, as shown in FIGS. 9A-9D, or with other devices such as TAVI.

FIGS. 9A-9D are cross-sectional schematics of the use of catheter delivery of gel microparticles to form a viscoelastic microparticle three-dimensional structure to repair peri-device leaks (FIG. 9A, minor PDL; FIG. 9B, small PDL; FIG. 9C, moderate PDL; FIG. 9D, large PDL; where small and moderate are associated with endovascular coils, endovascular plugs and large is associated with an LAA closure device.

Repair of Tissue Defects

The microparticles can be used to fill or occlude tissue defects or cavities left by surgical resection, such as following a tumor recission. In a preferred embodiment, fistulas are repaired by filling with microparticles, and, in some cases, subsequently replaced in whole or in part with the host's tissue.

External Magnet Steerable Catheters

In one embodiment, the catheter includes or is used with a soft printhead with omnidirectional steering capabilities using magnetic actuation, based on ferromagnetic soft materials with programmed magnetic polarities within the printhead device. The tip can include programmed ferromagnetic domains and hundreds-kilopascal-level rigidity, which can be quickly and reversibly deformed by applying static magnetic fields of 50˜200 mT, to enable active steering under remote magnetic manipulation. Depending on the required mechanical and magnetic properties, different types of soft polymer matrices (e.g. silicone elastomers or thermoplastic polyurethane) and ferromagnetic microparticles (e.g. neodymium iron boron, samarium cobalt) can be used to construct the ferromagnetic tips. When the tip has a uniform magnetization profile, the actuating domain will deflect along the applied magnetic field direction due to the torques generated from the embedded magnetic particles. Since this primary actuating domain follows the applied field direction, omnidirectional steering can be readily achieved with intuitive manipulation methods. This omnidirectional steerability is important for use with a transcatheter bioprinter to ensure that it can follow desired print paths in complex and constrained environments. To enable controlled magnetic manipulation, an actuation platform in the form of either a multi-axial electromagnetic device or a 6-DOF robotic arm holding/rotating a permanent magnet can be used to control the direction and strength of the applied actuation fields.

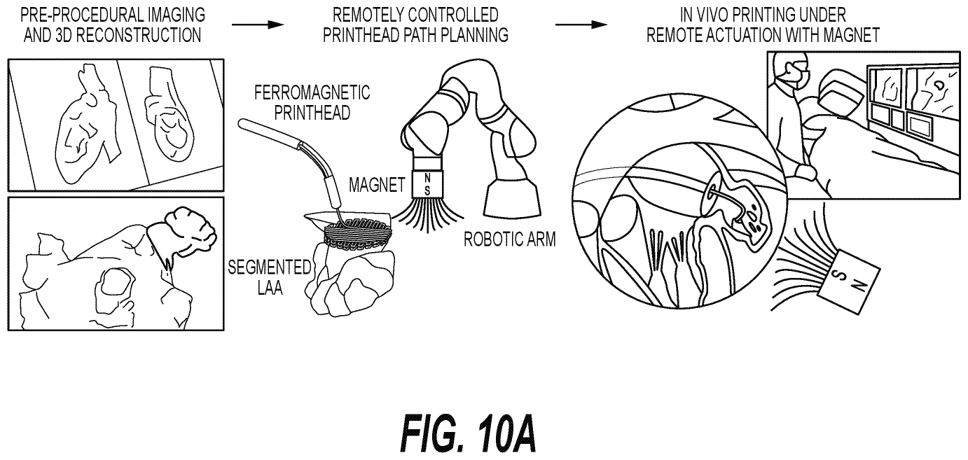

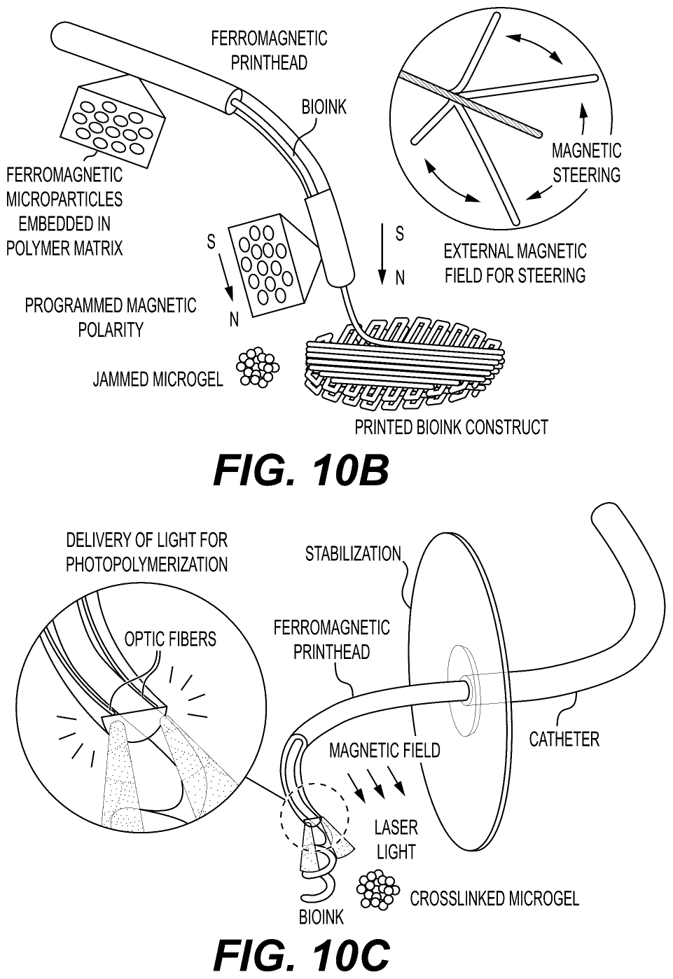

FIGS. 10A-10C are schematics of remotely steerable, ferromagnetic catheter printhead and use thereof. Step one is to conduct pre-procedural imaging and 3D reconstruction, then remotely controlled printhead path planning, then in vivo printing under remote actuation with a magnet (FIG. 10A). The ferromagnetic printhead typically includes a catheter tip having ferromagnetic particles embedded in a polymer matrix with programmed magnetic polarity. Steering of this printhead with an external magnet allows controlled printing of the jammed microgel. (FIG. 10B). Light delivered from optical fibers in the catheter provide a means for photopolymerization of the injected particles, sealant or ink solvent.

An advantage of the methods and materials described herein is that they allow for rapid, large volume delivery, for example, up to 25 mL, with timescales on the order of 15-60 seconds.

Creation of Robotic Fixture In Vivo

The technology can also be used with soft robotics approaches to build soft robotic elements in situ. For example, one can combine granular materials with different types of boundary layers/chambers to produce flexible grippers, actuators, and variable stiffness soft robots. If the chambers are collapsible, they can be loaded into catheters, navigated to a target tissue site, then the gel microparticles printed into the chambers to expand them and build the soft robotic element in situ using a fully-soft, minimally-invasive approach, rather than implanting it through an invasive approach with rigid tools. Soft grippers could be used to manipulate delicate tissue structures, and the actuation and variable stiffness could be used for programmed mechano-stimulation of target tissues.

### V. Kits

Kits are provided for use with the methods described above. In the simplest embodiment, the kit contains a vial of microparticles either in suspension or lyophilized for resuspension, typically with sterile water, for injection using a catheter. Alternatively, the kit would include a defined material formulation loaded in a sterile syringe which could then be attached to a catheter. Microparticles could come packaged in syringes of different volumes so the user could select which catheter and which vial of gel microparticles suits their needs.

In the preferred embodiment, the catheter/delivery tool would be provided separately. The user could add on the appropriate end effector (nozzle, needle, mesh, means for dispensing sealant) based on their needs. The catheter could have different “heads” based on the application.

For customization, the pre-loaded syringes could contain materials with different attributes (e.g., size, shape, mechanics), depending on the target application. This would avoid several steps by the end user and reduce the likelihood of improper rehydration of the particles, leading to an unknown viscoelastic state, formation of air gaps during syringe filling, loss of sterility during material transfer between components, and other potential problems.

Syringes/vials of hydrated (but viscoelastic) gel microparticles, rather than lyophilized particles, may reduce errors with rehydration. Pre-loading a syringe might also prevent gaps during filling and prevent loss of sterility.

If the stabilization is a distensible boundary mesh, this could be pre-loaded in the tip of the catheter.

In one embodiment, the process of using these materials would be:
order the appropriate kit for the target clinical application,
attach syringe to flushed catheter
navigate the catheter tip to the target site,
extrude the required amount of material for occlusion, typically confirmed with fluoroscopy,
withdraw the catheter and execute a self-sealing mechanism to close the mesh and prevent gel microparticles escape.

The mechanism could be a patch, a sealant, a photopolymerization step, a drawstring, or other means as described above.

For more complex approaches (e.g., spatial gradients, multi-material patterning, functionalization), the kit would include applicator tools such as double-barreled syringes with stopcocks/valves to switch from one to the other, or mixers that inject known/pre-determined volumes of different gel microparticles—delivery could be sequential, co-extrusion, coaxial extrusion, etc.

Modifications and variations of the materials and methods described herein are intended to be encompassed by the following claims.

We claim:

1. A minimally invasive method of creating a tissue filler, occluding agent, or tissue seal in a site in a patient in need thereof comprising
administering into the site a suspension of biocompatible hydrogel microparticles between about ten and 1000 microns in diameter using a catheter, syringe, or ink printer type device to apply the microparticles to form a viscoelastic hydrogel microparticle three-dimensional material at the site.

2. The method of claim 1 wherein the hydrogel microparticles have a diameter of between about 10 and 100 microns.

3. The method of claim 1 further comprising crosslinking or sealing with a tissue adhesive the three-dimensional material.

4. The method of claim 1 further comprising providing a support or encapsulating structure, preferably a surgical mesh, fabric, membrane, synthetic or biological matrix, at the time of implantation, prior to, with, or after administration of the microparticles.

5. The method of claim 4 wherein the microparticles are extruded with a support or encapsulating structure through a syringe or catheter or into the site to be treated containing a support or encapsulating structure.

6. The method of claim 5 wherein the mesh size prevents microparticle escape but may allow tissue integration, resulting in stabilization of the defect as cells infiltrate, using the microparticles and mesh as scaffold to form tissue to permanently repair the defect or occlude the structure.

7. The method of claim 1 wherein the microparticles are injected with force applied by gravity or applied pressure, preferably between 0.2 and 5 PSI, preferably wherein the microparticles suspension is in an unjammed state and subsequently stabilizes or self-assembles into a solid-like structure.

8. The method of claim 1 comprising administering the microparticles into a tissue defect, tissue tear, tissue lumen, appendage/outpouching or diseased tissue to form a three-dimensional viscoelastic hydrogel microparticle matrix.

9. The method of claim 1 comprising injecting the gel microparticles into cardiac anatomical features to form a three-dimensional structure occluding the left atrial appendage or repairing ventricular or atrial septal defect.

10. The method of claim 1 wherein the microparticles are administered to a vein or artery to fill or occlude a site to repair a vascular defect, such as a cerebral, aortic or peripheral aneurysm.

11. The method of claim 1 comprising injecting the gel microparticles to repair a post surgical or obstetrical defect.

12. The method of claim 1 wherein the microparticles are administered to form blockages to the passage of urine and fecal matter between the vagina or rectum or urethra.

13. The method of claim 1 wherein the microparticles are used to form a peri-device occlusion to prevent leakage post implantation of devices such as occluder devices, valves, stents, and flow diverters.

14. The method of claim 13 wherein the leaks are associated with endovascular coils, endovascular plugs, or transcatheter aortic valve implantation.

15. The method of claim 1 wherein microparticles are administered to form a homogenous structure, or a heterogeneous structure wherein microparticles having different composition and/or size are interspersed and/or layered.

16. The method of claim 1 wherein the microparticle matrix has sufficient interstitial spacing to allow cells to migrate into the matrix to form tissue.

17. The method of claim 1 comprising stabilizing the hydrogel microparticles by dispersing light through a light diffusing fiber tip to the top of the hydrogel microparticle structure to polymerize the gel.

18. The method of claim 1 comprising administering hydrogel microparticles using a catheter or cystoscope, optionally wherein the catheter is a multi-lumen catheter with multiple ports for attaching syringes or vials of microparticles/bioagents/sealants, wherein the catheter includes optic fibers in the catheter for delivering light, catheters including balloons on an end of the catheter for stabilization, or catheters including a suction device at an end for ensuring stabilization on tissue.

19. The method of claim 1 wherein the microparticles are administered using an external magnetically driven catheter.

20. A kit for use in the method of claim 1 comprising a vial or syringe containing microparticles either in suspension or lyophilized for resuspension, typically with sterile water.

21. The kit of claim 20 comprising a sterile syringe for attachment to a catheter.

22. The kit of claim 20 further comprising a catheter/delivery tool, optionally comprising effector ends such as a nozzle, needle, mesh, or means for dispensing sealant.

23. The kit of claim 20 comprising microparticles with different attributes such as size, shape, and mechanics, depending on the target application.

24. The kit of claim 20 comprising a distensible boundary encapsulating structure, optionally pre-loaded in the tip of a catheter.

25. A method of administering the materials in the kits of claim 20, comprising
providing a syringe containing the microparticles in a suspension,
attaching the syringe to a catheter,
navigating the catheter tip to the target site,
extruding the required amount of material for occlusion, preferably confirming with fluoroscopy, and
withdrawing the catheter and execute a self-sealing mechanism to close the mesh and prevent gel microparticles escape, preferably wherein the self-sealing mechanism is a patch, a sealant, a photopolymerization step, or a drawstring.
